SEQUENCE LISTING (14 sequences)

SEQ ID NO: 1 - DNA, 1698 bp, Chlamydia muridarum
  CDS (1)..(1698)
atgggattatctcgcctaattttatttggcttactttctttaccgctctcagcaagctgcgacttcccccccagtgtttcccagaagatattattcttgtgtcaaaaatctattcctcaagctctggagtcctatcttgaggcatctacaacctatcaacaacataacttttctatattgcgcttaatagctaagtcatacttacaacaaagtctcttttctgaagatgcttacgtacgcaaaagcgcaattattggagcggggctttctggctcatctgagactctagatctactgtctgaatccatagaaacacaggatctttatgagcagctacttattttaaatgctgcaggcaatcaattaggcaaaacttccgatcgtcttttattcaaaggattaacagcacctcatcctattattcgcttggaagctgcttaccgtctggcctgtatgaaaaacagtaaagtaagtgactacctctattcttttatccaccagcttccagaagaaatccaaaacttagcagcaacgatttttttgcagctcgaaacggaagaagcagatgcttatgttcatagactcctgtcttctcctaatagtctaacaagaaactatatggcttatctaattggagaatatcaacagaggagatttcttccaacgctccgctcgttgcttaccagcgcagctcctttagaccaagaaggatctttgtatgctataggaaaattagaagatgccagcagctatcctaaaatcaaagcattaagctccaaatctaaccctgaagtggctcttgctgctgctcagacattattattcttgggtaaagaagatgaggctcttcctatcctaactactttttgccagcaagagcttcctcgagctatttatacctctcgtttcctttcattagaaaaaggagaagagcttcttttacccatcttttgtaaagctattaaagaagaaattaaactgaatgctgctttggctcttgtccacttgggaagcgttaatcacctagtgcttagttatttaacagaatttttagaaaataaaattctccaccgcatatttttacccacccattcgataggaaaagccacgcagttttggaaagagtgtacggcactccctcttctaagcccagaagaaaaagcaagagctttggcaatgtatcgcgcagcagaagatacgatcctctctagtttattaaaattacctaacaatgcctatctgccttatttggaacgtattctaacttcacaaaaaacccctctagcagctaaagctattgcttttttatcagtaacagctcatcctcaggcactttctttagtctcgaaagcagcactaactccaggagaccctatcattcgcgcttatgcgaatttagctttatatacaatgacgcaagatcctgaaaagaaagccttattatatcaatatgccgaacagttaataggagacacgattttgtttacagatgaggagaatcccctgccttctccccattcttcctacctgcgatatcaagtgtccccagaaactcgttctcaactcatgctaactattttagaaaccctagtttcttctaaaactgatgaagacatccgagtttttctttcgctaatgaaaaaaacccattacaaaaatatccccatcttatctggattattaatgagaatagtggag

SEQ ID NO: 2 - protein, 566 aa, Chlamydia muridarum
MGLSRLILFGLLSLPLSASCDFPPSVSQKILFLCQKSIPQALESYLEASTTYQQHNFSILRLIAKSYLQQSLFSEDAYVRKSAIIGAGLSGSSETLDLLSESIETQDLYEQLLILNAAGNQLGKTSDRLLFKGLTAPHPIIRLEAAYRLACMKNSKVSDYLYSFIHQLPEEIQNLAATIFLQLETEEADAYVHRLLSSPNSLTRNYMAYLIGEYQQRRFLPTLRSLLTSAAPLDQEGSLYAIGKLEDASSYPKIKALSSKSNPEVALAAAQTLLFLGKEDEALPILTTFCQQELPRAIYTSRFLSLEKGEELLLPIFCKAIKEEIKLNAALALVHLGSVNHLVLSYLTEFLENKILHRIFLPTHSIGKATQFWKECTALPLLSPEEKARALAMYRAAEDTILSSLLKLPNNAYLPYLERILTSQKTPLAAKAIAFLSVTAHPQALSLVSKAALTPGDPIIRAYANLALYTMTQDPEKKALLYQYAEQLIGDTILFTDEENPLPSPHSSYLRYQVSPETRSQLMLTILETLVSSKTDEDIRVFLSLMKKTHYKNIPILSGLLMRIVE

SEQ ID NO: 3 - DNA, 1698 bp, Chlamydia trachomatis
  CDS (1)..(1698)
atgggactatctcgtctagccttcattagtttcctctcttttacactctcagccagctgtgattttccttcctcagtttctcagagaatcttgttttcttgccgaaaatcagtccctcaagctctagaagcctatctcgaagcttcagcaacttatcaacaacacgatttctccgtattacgcgtaatagcagaatcgtatttacaacaaagctttctctctgaggacacctacatacgtaaaagtgcaattattggagcagggctatctggttcatcagaagctttagagttactgtctgaggctatagaaacgcaagatctctatgagcaactactcattttaaatgctgcaaccagccaattaagcaaaacttctgacaaacttttattcaagggattaacagcttctcatcctgtcatccgcttagaagctgcttatcgtcttgcctgtatgaaaaatagcaaggtaagtgattacctttattcttttatctacaagttaccagaagaaattcaaaacctagcggcaactattttcttacaactcgaaacagaagaagctgatgcttatattcatcatttgctctcttctcccaataacctgacaagaaactatgttgcctatttaattggagagtacaaacaaaaaagatttcttccaacactacgctctttacttacaagtgcctctcctttagatcaagaaggcgctttgtatgcgttaggcaaactggaagactctggtagctatcctagaattaaagctctaagctctagatccaatcctgaagtagtactcgctgcagctcagacattattattcttagagaaagaagaagaagctctaccgatcctaaccaacctttgccaacaaaaacttcttcgagccctgtataccgcacgtttcctctcgcaagagaagggtgaagagcttcttcttccaatcttttataacgcaacacaagaagaaattagactgaatactgctttagcacttgttcatcaagggtgtacagatcctcaagtcctccactatctaacagaaatcttagaaagtaaagttctccatcgcatatttttacctactcactcgacaggaaaagctatacagttctggaaagaatgcaccacttttcctctcatgagccaagaagacaaaatgagaacgttggctatgtatcgggtagcggaagataccatcctctcagcgttactaaaattacccaatgacgcctatcttccttacctagagcgcatcctcgcctcacaaaaaactatactagcagctaaagctattgcttttttatcggtaacagctcatcctcaggcactttctttagtctcgaaagctgcattaactcctggagaccctatcattcgcgcttacgctaatctagctttatatacaatgaccaaagatcctgagaaaaaagctgtgctataccgatatgctgaacaattaatagaggataccattttattcacagatgctgaaaatccgcttccctctccaagctcttcttatttacgctaccaagtatcccctgagacccgcacacaacttatgctagctattttggaaaccttagtttcttccaaaacggatgaagatatccgcgtttttctttccctaatgaaaaaaacccattacaaaaatatcccgatcttatcaggattgttaatgagaatagtggag

SEQ ID NO: 4 - protein, 566 aa, Chlamydia trachomatis
MGLSRLAFISFLSFTLSASCDFPSSVSQRILFSCRKSVPQALEAYLEASATYQQHDFSVLRVIAESYLQQSFLSEDTYIRKSAIIGAGLSGSSEALELLSEAIETQDLYEQLLILNAATSQLSKTSDKLLFKGLTASHPVIRLEAAYRLACMKNSKVSDYLYSFIYKLPEEIQNLAATIFLQLETEEADAYIHHLLSSPNNLTRNYVAYLIGEYKQKRFLPTLRSLLTSASPLDQEGALYALGKLEDSGSYPRIKALSSRSNPEVVLAAAQTLLFLEKEEEALPILTNLCQQKLLRALYTARFLSQEKGEELLLPIFYNATQEEIRLNTALALVHQGCTDPQVLHYLTEILESKVLHRIFLPTHSTGKAIQFWKECTTFPLMSQEDKMRTLAMYRVAEDTILSALLKLPNDAYLPYLERILASQKTILAAKAIAFLSVTAHPQALSLVSKAALTPGDPIIRAYANLALYTMTKDPEKKAVLYRYAEQLIEDTILFTDAENPLPSPSSSYLRYQVSPETRTQLMLAILETLVSSKTDEDIRVFLSLMKKTHYKNIPILSGLLMRIVE

SEQ ID NO: 5 - DNA, 1695 bp, Chlamydia muridarum
  CDS (1)..(1695)
atgtgcgacttcccccccagtgtttcccagaagatattattcttgtgtcaaaaatctattcctcaagctctggagtcctatcttgaggcatctacaacctatcaacaacataacttttctatattgcgcttaatagctaagtcatacttacaacaaagtctcttttctgaagatgcttacgtacgcaaaagcgcaattattggagcggggctttctggctcatctgagactctagatctactgtctgaatccatagaaacacaggatctttatgagcagctacttattttaaatgctgcaggcaatcaattaggcaaaacttccgatcgtcttttattcaaaggattaacagcacctcatcctattattcgcttggaagctgcttaccgtctggcctgtatgaaaaacagtaaagtaagtgactacctctattcttttatccaccagcttccagaagaaatccaaaacttagcagcaacgatttttttgcagctcgaaacggaagaagcagatgcttatgttcatagactcctgtcttctcctaatagtctaacaagaaactatatggcttatctaattggagaatatcaacagaggagatttcttccaacgctccgctcgttgcttaccagcgcagctcctttagaccaagaaggatctttgtatgctataggaaaattagaagatgccagcagctatcctaaaatcaaagcattaagctccaaatctaaccctgaagtggctcttgctgctgctcagacattattattcttgggtaaagaagatgaggctcttcctatcctaactactttttgccagcaagagcttcctcgagctatttatacctctcgtttcctttcattagaaaaaggagaagagcttcttttacccatcttttgtaaagctattaaagaagaaattaaactgaatgctgctttggctcttgtccacttgggaagcgttaatcacctagtgcttagttatttaacagaatttttagaaaataaaattctccaccgcatatttttacccacccattcgataggaaaagccacgcagttttggaaagagtgtacggcactccctcttctaagcccagaagaaaaagcaagagctttggcaatgtatcgcgcagcagaagatacgatcctctctagtttattaaaattacctaacaatgcctatctgccttatttggaacgtattctaacttcacaaaaaacccctctagcagctaaagctattgcttttttatcagtaacagctcatcctcaggcactttctttagtctcgaaagcagcactaactccaggagaccctatcattcgcgcttatgcgaatttagctttatatacaatgacgcaagatcctgaaaagaaagccttattatatcaatatgccgaacagttaataggagacacgattttgtttacagatgaggagaatcccctgccttctccccattcttcctacctgcgatatcaagtgtccccagaaactcgttctcaactcatgctaactattttagaaaccctagtttcttctaaaactgatgaagacatccgagtttttctttcgctaatgaaaaaaacccattacaaaaatatccccatcttatctggattattaatgagaatagtggagcgagctcggtaccaagcttacgtagaacaaaaactcatctcagaagaggat

SEQ ID NO: 6 - protein, 565 aa, Chlamydia muridarum
MCDFPPSVSQKILFLCQKSIPQALESYLEASTTYQQHNFSILRLIAKSYLQQSLFSEDAYVRKSAIIGAGLSGSSETLDLLSESIETQDLYEQLLILNAAGNQLGKTSDRLLFKGLTAPHPIIRLEAAYRLACMKNSKVSDYLYSFIHQLPEEIQNLAATIFLQLETEEADAYVHRLLSSPNSLTRNYMAYLIGEYQQRRFLPTLRSLLTSAAPLDQEGSLYAIGKLEDASSYPKIKALSSKSNPEVALAAAQTLLFLGKEDEALPILTTFCQQELPRAIYTSRFLSLEKGEELLLPIFCKAIKEEIKLNAALALVHLGSVNHLVLSYLTEFLENKILHRIFLPTHSIGKATQFWKECTALPLLSPEEKARALAMYRAAEDTILSSLLKLPNNAYLPYLERILTSQKTPLAAKAIAFLSVTAHPQALSLVSKAALTPGDPIIRAYANLALYTMTQDPEKKALLYQYAEQLIGDTILFTDEENPLPSPHSSYLRYQVSPETRSQLMLTILETLVSSKTDEDIRVFLSLMKKTHYKNIPILSGLLMRIVERARYQAYVEQKLISEED

SEQ ID NO: 7 - DNA, 1644 bp, Chlamydia trachomatis
  CDS (1)..(1644)
atgtgtgattttccttcctcagtttctcagagaatcttgttttcttgccgaaaatcagtccctcaagctctagaagcctatctcgaagcttcagcaacttatcaacaacacgatttctccgtattacgcgtaatagcagaatcgtatttacaacaaagctttctctctgaggacacctacatacgtaaaagtgcaattattggagcagggctatctggttcatcagaagctttagagttactgtctgaggctatagaaacgcaagatctctatgagcaactactcattttaaatgctgcaaccagccaattaagcaaaacttctgacaaacttttattcaagggattaacagcttctcatcctgtcatccgcttagaagctgcttatcgtcttgcctgtatgaaaaatagcaaggtaagtgattacctttattcttttatctacaagttaccagaagaaattcaaaacctagcggcaactattttcttacaactcgaaacagaagaagctgatgcttatattcatcatttgctctcttctcccaataacctgacaagaaactatgttgcctatttaattggagagtacaaacaaaaaagatttcttccaacactacgctctttacttacaagtgcctctcctttagatcaagaaggcgctttgtatgcgttaggcaaactggaagactctggtagctatcctagaattaaagctctaagctctagatccaatcctgaagtagtactcgctgcagctcagacattattattcttagagaaagaagaagaagctctaccgatcctaaccaacctttgccaacaaaaacttcttcgagccctgtataccgcacgtttcctctcgcaagagaagggtgaagagcttcttcttccaatcttttataacgcaacacaagaagaaattagactgaatactgctttagcacttgttcatcaagggtgtacagatcctcaagtcctccactatctaacagaaatcttagaaagtaaagttctccatcgcatatttttacctactcactcgacaggaaaagctatacagttctggaaagaatgcaccacttttcctctcatgagccaagaagacaaaatgagaacgttggctatgtatcgggtagcggaagataccatcctctcagcgttactaaaattacccaatgacgcctatcttccttacctagagcgcatcctcgcctcacaaaaaactatactagcagctaaagctattgcttttttatcggtaacagctcatcctcaggcactttctttagtctcgaaagctgcattaactcctggagaccctatcattcgcgcttacgctaatctagctttatatacaatgaccaaagatcctgagaaaaaagctgtgctataccgatatgctgaacaattaatagaggataccattttattcacagatgctgaaaatccgcttccctctccaagctcttcttatttacgctaccaagtatcccctgagacccgcacacaacttatgctagctattttggaaaccttagtttcttccaaaacggatgaagatatccgcgtttttctttccctaatgaaaaaaacccattacaaaaatatcccgatcttatcaggattgttaatgagaatagtggag

SEQ ID NO: 8 - protein, 548 aa, Chlamydia trachomatis
MCDFPSSVSQRILFSCRKSVPQALEAYLEASATYQQHDFSVLRVIAESYLQQSFLSEDTYIRKSAIIGAGLSGSSEALELLSEAIETQDLYEQLLILNAATSQLSKTSDKLLFKGLTASHPVIRLEAAYRLACMKNSKVSDYLYSFIYKLPEEIQNLAATIFLQLETEEADAYIHHLLSSPNNLTRNYVAYLIGEYKQKRFLPTLRSLLTSASPLDQEGALYALGKLEDSGSYPRIKALSSRSNPEVVLAAAQTLLFLEKEEEALPILTNLCQQKLLRALYTARFLSQEKGEELLLPIFYNATQEEIRLNTALALVHQGCTDPQVLHYLTEILESKVLHRIFLPTHSTGKAIQFWKECTTFPLMSQEDKMRTLAMYRVAEDTILSALLKLPNDAYLPYLERILASQKTILAAKAIAFLSVTAHPQALSLVSKAALTPGDPIIRAYANLALYTMTKDPEKKAVLYRYAEQLIEDTILFTDAENPLPSPSSSYLRYQVSPETRTQLMLAILETLVSSKTDEDIRVFLSLMKKTHYKNIPILSGLLMRIVE

SEQ ID NO: 9 - DNA, 41 bp, Chlamydia trachomatis
ataagaatgcggccgccaccatgtgcgacttcccccccagt

SEQ ID NO: 10 - DNA, 40 bp, Chlamydia trachomatis
gttggtaccgagctcgctccactattctcattaataatcc

SEQ ID NO: 11 - DNA, 41 bp, Chlamydia trachomatis; identical to SEQ ID NO: 9

SEQ ID NO: 12 - DNA, 40 bp, Chlamydia trachomatis; identical to SEQ ID NO: 10

SEQ ID NO: 13 - DNA, 31 bp, Chlamydia trachomatis
gaattcggatccgatgggattatctcgccta

SEQ ID NO: 14 - DNA, 36 bp, Chlamydia trachomatis
attaagaatgcggccgctttatcactccactattct

(19) United States
(12) Patent Application Publication
(10) Pub. No.: US 2008/0166376 A1
(43) Pub. Date: Jul. 10, 2008

(54) Immunization Against Chlamydia Infection

(75) Inventors: Robert Brunham, Vancouver (CA); Scott Gallichan, Campbellville (CA); Andrew Murdin, Richmond Hill (CA); Ausra Raudonikiene, Toronto (CA)

Correspondence Address: Patrick J Halloran, 3141 Muirfiled Rd, Center Valley, PA, 18034, US

(73) Assignee: B/E INTELLECTUAL PROPERTY, LENEXA KANSAS (KR)

(21) Appl. No.: 10/580,142

(22) PCT Filed: Nov. 19, 2004
(86) PCT No.: PCT/CA04/02001

§ 371 Date: Oct. 10, 2007

Related U.S. Application Data
(60) Provisional application No. 60/481,690, filed on Nov. 21, 2003.

(51) Int. Cl. A61K 39/118; C07H 21/04; A61K 31/711; C12N 1/00; C07K 14/295; C12P 21/06; C07K 16/12; A61K 38/16

(52) U.S. Cl. 424/263.1; 536/23.7; 536/23.4; 514/44; 435/243; 536/24.3; 530/350; 435/69.1; 530/387.9; 514/12

(57) ABSTRACT

The present invention provides nucleic acids, proteins and vectors for a method of nucleic acid, including DNA, immunization of a host, including humans, against disease caused by infection by a strain of Chlamydia, specifically C. trachomatis. The method employs a vector containing a nucleotide sequence encoding a Mgp002 polypeptide of a strain of Chlamydia operably linked to a promoter to effect expression of the gene product in the host. Truncated forms of the full-length Mgp002 gene are useful immunogens for protecting against disease caused by infection with Chlamydia. The invention further provides recombinant Mgp002 protein useful for protecting against disease caused by infection with Chlamydia.

### FIELD OF INVENTION

The present invention relates to immunology and, in particular, to immunization of hosts using nucleic acid molecules to provide protection against infection by Chlamydia.

### BACKGROUND OF THE INVENTION

Nucleic acid immunization is an approach for generating protective immunity against infectious diseases. (ref. 1—throughout this application, various references are cited in parentheses to describe more fully the state of the art to which this invention pertains. (Full bibliographic information for each citation is found at the end of the specification, immediately preceding the claims. The disclosure of these references are hereby incorporated by reference into the present disclosure). Unlike protein or peptide based subunit vaccines, nucleic acid or DNA immunization provides protective immunity through expression of foreign proteins by host cells, thus allowing the presentation of antigen to the immune system in a manner more analogous to that which occurs during infection with viruses or intracellular pathogens (ref. 2). Although considerable interest has been generated by this technique, successful immunity has been most consistently induced by DNA immunization for viral diseases (ref. 3). Results have been more variable with non-viral pathogens which may reflect differences in the nature of the pathogens, in the immunizing antigens chosen, and in the routes of immunization (ref. 4). Further development of DNA vaccination will depend on elucidating the underlying immunological mechanisms and broadening its application to other infectious diseases for which existing strategies of vaccine development have failed.

The genus Chlamydia includes four species, Chlamydia trachomatis, C. pneumoniae, C. psittaci and C. pecorum. Chlamydia trachomatis is an obligate intracellular bacterial pathogen which usually remains localized to mucosal epithelial surfaces of the human host. Chlamydiae are dimorphic bacteria with an extracellular spore-like transmission cell termed the elementary body (EB) and an intracellular replicative cell termed the reticulate body (ref. 5). C. trachomatis is one of the most common sexually transmitted pathogens and the main cause of preventative blindness worldwide (ref. 6). From a public health perspective, chlamydial infections are of great importance because they are significant causes of infertility, blindness and are a prevalent co-factor facilitating the transmission of human immunodeficiency virus type 1 (ref. 7). There are multiple serovars of C. trachomatis that cause trachoma, genital, respiratory and ocular infections. Protective immunity to C. trachomatis is thought to be effected through T-cell-mediated immunity by cytokine's released by Thl-like CD 4 lymphocyte responses and by local antibody in mucosal secretions and is believed to be primarily directed to the major outer membrane protein (MOMP), which is quantitatively the dominant surface protein on the chlamydial bacterial cell and has a molecular mass of about 40 kDa (ref. 11). The role of CD8+ T-cells appears to be secondary.

Initial efforts in developing a chlamydial vaccine were based on parenteral immunization with the whole bacterial cell. Although this approach met with some success in human trials, it was limited because protection was short-lived, partial and vaccination may exacerbate disease during subsequent infection episodes possibly due to pathological reactions to certain chlamydial antigens (ref. 8). More recent attempts at chlamydial vaccine design have been based on a subunit design using MOMP protein or peptides (ref 9). These subunit vaccines have also generally failed, perhaps because the immunogens do not induce protective cellular and humoral immune responses recalled by native epitopes on the organism (ref. 10).

In U.S. Pat. No. 6,235,290 filed Jul. 11, 1997, assigned to University of Manitoba and the disclosure of which is incorporated herein by reference, the generation of a protective immune response using a DNA sequence which encodes the MOMP of C. trachomatis in a plasmid by DNA immunization have been described.

Recently both the Chlamydia trachomatis (ref 14) and the C. muridium (ref 15) mouse pneumonitis strain (MoPn) entire genomes have been sequenced. The mgp002 gene from Chlamydia pneumonia was disclosed in PCT publication WO01/21803 published on 29 Mar. 2001.

Chlamydial infections may be treated with antibiotics, such as tetracycline derivatives, especially doxycycline, and the macrolide or azalides such as erythromycin and azithromycin; however, infections are often asymptomatic, with severe complications usually presenting as the first symptoms of an infection (ref 6). Chemotherapeutic or antibiotic therapy may not be a viable long-term strategy as increasing use of antibiotics have led to the increase in antibiotic resistant micro-organisms. Thus, there remains the need for effective therapies for preventing and treating chlamydial infections.

### SUMMARY OF THE INVENTION

The present invention is concerned with nucleic acid immunization, specifically DNA immunization, to generate in a host a protective immune response to a Mgp002 gene or a truncated from thereof of a strain of Chlamydia.

Accordingly, in one aspect, the present invention provides a nucleic acid molecule comprising a nucleic acid sequence which encodes a polypeptide selected from any one of: (a) SEQ ID No: 2; (b) SEQ ID No: 4; (c) SEQ ID No: 6 (d) SEQ ID No: 8 (e) an immunogenic fragment comprising at least 12 consecutive amino acids from a polypeptide of (a) to (d); and (f) a polypeptide of (a), (b) (c) or (d) which has been modified by conservative amino acid substitution without loss of immunogenicity, wherein said modified polypeptide is at least 75% identical in amino acid sequence to the corresponding polypeptide of (a), (b) (c) or (d).

In a further aspect of the present invention, there is provided a nucleic acid molecule comprising a nucleic acid sequence which encodes a polypeptide selected from any one of: (a) SEQ ID No: 2; (b) SEQ ID No: 4; (c) SEQ ID No: 6 (d) SEQ ID No: 8 (e) an immunogenic fragment comprising at least 12 consecutive amino acids from a polypeptide of (a) to (d); and (f) a polypeptide of (a), (b), (c) or (d) which has been modified by conservative amino acid substitution without loss of immunogenicity, wherein said modified polypeptide is at least 75% identical in amino acid sequence to the corresponding polypeptide of (a), (b) (c) or (d) wherein said nucleic acid molecule is operatively coupled to a sequence for expression of said nucleic acid molecule in a host to which the nucleic acid molecule is administered.

The sequence for expression may be a cytomegalovirus promoter, and may be contained in the human cytomegalovirus major immediate-early promoter-enhancer region. Other suitable promoters can be viral promoter or other mammalian promoters that are capable of promoting expression in a target eukaryotic cell. The vector may be a plasmid vector and the nucleotide sequence may be that of SEQ ID No: 1, 3, 5 or 7.

The strain of Chlamydia may be a strain or serovar of Chlamydia including Chlamydia trachomatis or Chlamydia pneumoniae. The non-replicating vector may be plasmid pcDNA3.1 into which the nucleotide sequence is inserted or a derivative or modification thereof.

In a further aspect of the present invention, there is provided an immunogenic composition for in vivo administration to a host for the generation in the host of a protective immune response to a Mgp002 gene or a fragment thereof, of a strain of Chlamydia, comprising a non-replicating vector as provided herein and a pharmaceutically-acceptable carrier therefor.

In a further aspect of the invention there is provided An isolated polynucleotide from a strain of Chlamydia selected from the group consisting of: a polynucleotide comprising the nucleotide sequence of SEQ ID NO:1; a polynucleotide comprising the nucleotide sequence of SEQ ID NO:3; a polynucleotide comprising the nucleotide sequence of SEQ ID NO:5; a polynucleotide comprising the nucleotide sequence of SEQ ID NO:7; a polynucleotide that is at least 95% homologous to the nucleotide sequence of SEQ ID NO:1, 3, 5, or 7; and a polynucleotide which hybridizes under stringent hybridizing conditions of 6×SSC containing 50% formamide at 42° C. with a polynucleotide comprising the nucleotide sequence of SEQ ID NO:1, 3, 5, or 7, wherein administration of said isolated polynucleotide, in an immunogenically-effective amount to a mammal, induces an immune response in said mammal against infection by said strain of Chlamydia.

In an additional aspect of the invention, there is provided a vaccine comprising a vector comprising a nucleic acid molecule which encodes a polypeptide selected from any one of: (a) SEQ ID No: 2; (b) SEQ ID No. 4; (c) SEQ ID No: 6 (d) SEQ ID No: 8 (e) an immunogenic fragment comprising at least 100 consecutive amino acids from the polypeptide of any one of (a) to (d); and (f) a polypeptide of any one of (a) to (e) which has been modified by conservative amino acid substitution, wherein said modified polypeptide is at least 90% identical in amino acid sequence to the corresponding polypeptide of any one of (a) to (e); wherein the nucleic acid molecule is either operatively linked to one or more control sequences for expression of the polypeptide in a mammalian or a bacterial cell, wherein the vaccine provides an immune response protective against disease caused by Chlamydia.

In a further aspect of the invention, there is provided A pharmaceutical composition comprising a pharmaceutically acceptable carrier or diluent suitable for use in a vaccine and a nucleic acid molecule which encodes a polypeptide selected from any one of: (a) SEQ ID No: 2; (b) SEQ ID No. 4; (c) SEQ ID No: 6 (d) SEQ ID No: 8 (e) an immunogenic fragment comprising at least 100 consecutive amino acids from the polypeptide of (a) to (d); and (f) a polypeptide of any one of (a) to (e) which has been modified by conservative amino acid substitution without loss of immunogenicity; wherein said modified polypeptide is at least 90% identical in amino acid sequence to the corresponding polypeptide of any one of (a) to (e); wherein the nucleic acid molecule is operatively linked to one or more control sequences for expression of the polypeptide in a mammalian cell.

In an additional aspect of the invention, there is provided a method of immunizing a host against disease caused by infection with a strain of Chlamydia, which comprises administering to said host an effective amount of a non-replicating vector as provided herein.

The nucleic acid molecule may be administered to the host, including a human host, in any convenient manner, such as intramuscularly or intranasally.

In an additional aspect of the invention, there is provided a method for preventing or treating Chlamydia infection comprising the step of administering an effective amount of a nucleic acid molecule which encodes a polypeptide selected from any one of: (a) SEQ ID No: 2; (b) SEQ ID No. 4; (c) an immunogenic fragment comprising at least 100 consecutive amino acids from the polypeptide of (a) to (c); and (d) a polypeptide of any one of (a) to (c) which has been modified by conservative amino acid substitution without loss of immunogenicity, wherein said modified polypeptide is at least 90% identical in amino acid sequence to the corresponding polypeptide of any one of (a) to (c); wherein the nucleic acid molecule is operatively linked to one or more control sequences for expression of the polypeptide.

The various options and alternatives discussed above may be employed in this aspect of the invention.

Those skilled in the art will readily understand that the invention, having provided the polynucleotide sequences encoding Chlamydia polypeptides, also provides polynucleotides encoding fragments derived from such polypeptides. Moreover, the invention is understood to provide mutants and derivatives of such polypeptides and fragments derived therefrom, which result from the addition, deletion, or substitution of non-essential amino acids as described herein. Those skilled in the art would also readily understand that the invention, having provided the polynucleotide sequences encoding Chlamydia polypeptides, further provides monospecific antibodies that specifically bind to such polypeptides.

The present invention has wide application and includes expression cassettes, vectors, and cells transformed or transfected with the polynucleotides of the invention.

### BRIEF DESCRIPTION OF DRAWINGS

The present invention will further be understood from the following description with reference to the drawings in which:

FIG. 1 shows the full-length nucleotide sequence of the Mgp002 gene (SEQ ID No: 1) and the deduced amino acid sequence of the full-length Mgp002 gene product (SEQ ID No:2) from Chlamydia muridium (strain Nigg) as well as the signal sequence deleted nucleotide sequence (starting at arrow) (SEQ ID No:5) and the deduced amino acid sequence (SEQ ID No:6).

FIG. 2 shows the full-length nucleotide sequence of the Mgp002 gene (SEQ ID No: 3) and the deduced amino acid sequence of the full-length Mgp002 gene product SEQ ID No:4) as well as the signal sequence deleted nucleotide sequence (starting at arrow) (SEQ ID No:7) and the deduced amino acid sequence (SEQ ID No:8) from Chlamydia trachomatis (serovar D).

FIG. 3 shows a schematic representation of one embodiment of the immunization protocol for treating chlamydial infection with a nucleic acid molecule encoding a Mgp002 gene or truncated form thereof. IM refers to intramuscular immunization while IN refers to intra nasal immunization.

FIG. 4, comprising panels A and B, show the results of immunization with a nucleic acid molecule encoding a full-length Mgp002 gene (Panel A) and a signal-sequence deleted Mgp002 gene (Panel B), cloned into plasmid pcDNA3.1, on the body weight loss in immunized Balb/c mice challenged with infectious chlamydia. Legend: EB=host-killed elementary bodies, PCACTmgp002=pcDNA3 with full-length Mgp002 gene inserted, PCACTmgp002delta=signal sequence deleted Mgp002 gene, naïve=no immunization, pAMycHis=empty vector.

FIG. 5, comprising panels A and B, shows the results of enhanced clearance of Chlamydia from the lungs of Balb/c mice immunized with a full-length Mgp002 gene (Panel A) and a signal-sequence deleted Mgp002 gene (Panel B) and challenged with infectious chlamydia. Legend: EB=host-killed elementary bodies, PCACTmgp002=pcDNA3 with full-length Mgp002 gene inserted, PCACTmgp002delta=signal sequence deleted Mgp002 gene, naïve=no immunization, pAMycHis=empty vector.

FIG. 6, illustrates graphically the construction of a plasmid, pET30b(+)mgp002+SP, for the expression of recombinant Mgp002 protein that contains a N-terminal His-Tag®.

FIG. 7, graphically illustrates the protection from genital challenge with Chlamydia trachomatis serovar D in CH3 mice immunized with purified recombinant Mgp002 protein with an ISCOM adjuvant. Animals were immunized subcutaneously with either saline (Naïve) Mgp002 protein (mgp002) or Chlamydia elimentary bodies (EB) and then challenged subsequently intravaginally with live Chlamydia trachomatis serovar D. Infectious units of Chlamydia were determined from washes at day 3 and 5 post infection.

### DETAILED DESCRIPTION OF THE INVENTION

To illustrate the present invention, plasmid DNA was constructed containing a nucleic acid molecule encoding Mgp002 gene from the C. trachomatis mouse pneumonitis strain (MoPn), which is a natural murine pathogen, permitting experimentation to be effected in mice. It is known that primary infection in the mouse model induces strong protective immunity to reinfection. For human immunization, a nucleic acid molecule encoding Mgp002 gene or a truncated form thereof of Chlamydia trachomatis can be used.

Any convenient plasmid vector may be used, such as pcDNA3.1, a eukaryotic II-selectable expression vector (Invitrogen, San Diego, Calif., USA), containing a human cytomegalovirus major-immediate-early promoter-enhancer region or a derivative thereof such as pCAMycHis. The nucleic acid molecule encoding Mgp002 gene or fragment thereof, may be inserted in the vector in any convenient manner. The gene may be amplified from Chlamydia trachomatis genomic DNA by PCR using suitable primers and the PCR product cloned into the vector. The nucleic acid molecule encoding Mgp002 gene or fragment thereof gene-carrying plasmid may be transferred, such as by electroporation, into E. coli or any suitable host for replication therein. Plasmids may be extracted from the E. coli in any convenient manner.

According to a first aspect of the invention, isolated polynucleotides are provided which encode Chlamydia polypeptides, whose amino acid sequences are shown in SEQ ID Nos: 2, 4, 6 and 8.

The term “isolated polynucleotide” is defined as a polynucleotide removed from the environment in which it naturally occurs. For example, a naturally-occurring DNA molecule present in the genome of a living bacteria or as part of a gene bank is not isolated, but the same molecule separated from the remaining part of the bacterial genome, as a result of, e.g. a cloning event (amplification), is isolated. Typically, an isolated DNA molecule is free from DNA regions (e.g., coding regions) with which it is immediately contiguous at the 5′ or 3′ end, in the naturally occurring genome. Such isolated polynucleotides may be part of a vector or a composition and still be defined as isolated in that such a vector or composition is not part of the natural environment of such polynucleotide.

The polynucleotide of the invention is either RNA or DNA (cDNA, genomic DNA, or synthetic DNA), or modifications, variants, homologs or fragments thereof. The DNA is either double-stranded or single-stranded, and, if single-stranded, is either the coding strand or the non-coding (anti-sense) strand. Any one of the sequences that encode the polypeptides of the invention as shown in SEQ ID No: 1, 3, 5 and 7 are (a) a coding sequence, (b) a ribonucleotide sequence derived from transcription of (a), or (c) a coding sequence which uses the redundancy or degeneracy of the genetic code to encode the same polypeptides. By “polypeptide” or “protein” is meant any chain of amino acids, regardless of length or post-translational modification (e.g., glycosylation or phosphorylation). Both terms are used interchangeably in the present application.

Consistent with the first aspect of the invention, amino acid sequences are provided which are homologous to SEQ ID No: 2, 4, 6 or 8. As used herein, “homologous amino acid sequence” is any polypeptide which is encoded, in whole or in part, by a nucleic acid sequence which hybridizes at 25-35° C. below critical melting temperature (Tm), to any portion of the nucleic acid sequence of SEQ ID No: 1, 3, 5 or 7. A homologous amino acid sequence is one that differs from an amino acid sequence shown in SEQ ID No: 2, 4, 6 or 8 by one or more conservative amino acid substitutions. Such a sequence also encompass serotypic variants (defined below) as well as sequences containing deletions or insertions which retain inherent characteristics of the polypeptide such as immunogenicity. Preferably, such a sequence is at least 75%, more preferably 80%, and most preferably 90% to 95% identical to SEQ ID No: 2, 4, 6 or 8.

Homologous amino acid sequences include sequences that are identical or substantially identical to SEQ ID No: 2, 4, 6 or 8. By “amino acid sequence substantially identical” is meant a sequence that is at least 90%, preferably 95%, more preferably 97%, and most preferably 99% identical to an amino acid sequence of reference and that preferably differs from the sequence of reference by a majority of conservative amino acid substitutions.

Conservative amino acid substitutions are substitutions among amino acids of the same class. These classes include, for example, amino acids having uncharged polar side chains, such as asparagine, glutamine, serine, threonine, and tyrosine; amino acids having basic side chains, such as lysine, arginine, and histidine; amino acids having acidic side chains, such as aspartic acid and glutamic acid; and amino acids having nonpolar side chains, such as glycine, alanine, valine, leucine, isoleucine, proline, phenylalanine, methionine, tryptophan, and cysteine.

Homology is measured using sequence analysis software such as Sequence Analysis Software Package of the Genetics Computer Group, University of Wisconsin Biotechnology Center, 1710 University Avenue, Madison, Wis. 53705. Amino acid sequences are aligned to maximize identity. Gaps may be artificially introduced into the sequence to attain proper alignment. Once the optimal alignment has been set up, the degree of homology is established by recording all of the positions in which the amino acids of both sequences are identical, relative to the total number of positions.

Homologous polynucleotide sequences are defined in a similar way. Preferably, a homologous sequence is one that is at least 45%, more preferably 60%, and most preferably 85% identical to the coding sequence of SEQ ID No: 1, 3, 5 or 7.

Consistent with the first aspect of the invention, polypeptides having a sequence homologous to SEQ ID No: 2, 4, 6 or 8 include naturally-occurring allelic variants, as well as mutants or any other non-naturally occurring variants that retain the inherent characteristics of the polypeptide of SEQ ID No: 2, 4, 6 or 8.

As is known in the art, an allelic variant is an alternate form of a polypeptide that is characterized as having a substitution, deletion, or addition of one or more amino acids that does not alter the biological function of the polypeptide. By “biological function” is meant the function of the polypeptide in the cells in which it naturally occurs, even if the function is not necessary for the growth or survival of the cells. For example, the biological function of a portion is to allow the entry into cells of compounds present in the extracellular medium. Biological function is distinct from antigenic property. A polypeptide can have more than one biological function. Different allelic variants may have the similar antigenic properties.

Allelic variants are very common in nature. For example, a bacterial species such as C. trachomatis is usually represented by a variety of serovars that differ from each other by minor allelic variations. Indeed, a polypeptide that fulfills the same biological function in different strains can have an amino acid sequence (and polynucleotide sequence) that is not identical in each of the strains. Despite this variation, an immune response directed generally against many allelic variants has been demonstrated. In studies of the Chlamydial MOMP antigen, cross-strain antibody binding plus neutralization of infectivity occurs despite amino acid sequence variation of MOMP from strain to strain, indicating that the MOMP, when used as an immunogen, is tolerant of amino acid variations.

Polynucleotides encoding homologous polypeptides or allelic variants are retrieved by polymerase chain reaction (PCR) amplification of genomic bacterial DNA extracted by conventional methods. This involves the use of synthetic oligonucleotide primers matching upstream and downstream of the 5′ and 3′ ends of the encoding domain. Suitable primers are designed according to the nucleotide sequence information provided in SEQ ID No: 1, 3, 5 or 7. The procedure is as follows: a primer is selected which consists of 10 to 40, preferably 15 to 25 nucleotides. It is advantageous to select primers containing C and G nucleotides in a proportion sufficient to ensure efficient hybridization; i.e., an amount of C and G nucleotides of at least 40%, preferably 50% of the total nucleotide content. A standard PCR reaction contains typically 0.5 to 5 Units of Taq DNA polymerase per 100 μL, 20 to 200 μM deoxynucleotide each, preferably at equivalent concentrations, 0.5 to 2.5 mM magnesium over the total deoxynucleotide concentration, 10⁵ to 10⁶ target molecules, and about 20 pmol of each primer. About 25 to 50 PCR cycles are performed, with an annealing temperature 15° C. to 5° C. below the true Tm of the primers. A more stringent annealing temperature improves discrimination against incorrectly annealed primers and reduces incorporation of incorrect nucleotides at the 3′ end of primers. A denaturation temperature of 95° C. to 97° C. is typical, although higher temperatures may be appropriate for dematuration of G+C-rich targets. The number of cycles performed depends on the starting concentration of target molecules, though typically more than 40 cycles is not recommended as non-specific background products tend to accumulate.

An alternative method for retrieving polynucleotides encoding homologous polypeptides or allelic variants is by hybridization screening of a DNA or RNA library. Hybridization procedures are well-known in the art. Important parameters for optimizing hybridization conditions are reflected in a formula used to obtain the critical melting temperature above which two complementary DNA strands separate from each other. For polynucleotides of about 600 nucleotides or larger, this formula is as follows: Tm 81.5+0.41×(% G+C)+16.6 log(cation ion concentration)−0.63×(% formamide)−600/base number. Under appropriate stringency conditions, hybridization temperature (Th) is approximately 20 to 40° C., 20 to 25° C., or, preferably 30 to 40° C. below the calculated Tm. Those skilled in the art will understand that optimal temperature and salt conditions can be readily determined.

For the polynucleotides of the invention, stringent conditions are achieved for both pre-hybridizing and hybridizing incubations (i) within 4-16 hours at 42° C., in 6×SSC containing 50% formamide, or (ii) within 4-16 hours at 65° C. in an aqueous 6×SSC solution (1 M NaCJ, 0.1 M sodium citrate (pH 7.0)). Typically, hybridization experiments are performed at a temperature from 60 to 68° C., e.g. 65° C. At such a temperature, stringent hybridization conditions can be achieved in 6×SSC, preferably in 2×SSC or 1×SSC, more preferably in 0.5×SSc, 0.3×SSC or 0.1×SSC (in the absence of formamide). 1×SSC contains 0.15 M NaCl and 0.015 M sodium citrate. Those skilled in the art will understand that the probe nucleic acid sequence will hybridize to the complimentary target nucleic acid sequence.

Useful homologs and fragments thereof that do not occur naturally are designed using known methods for identifying regions of an antigen that are likely to tolerate amino acid sequence changes and/or deletions. As an example, homologous polypeptides from different species are compared; conserved sequences are identified. The more divergent sequences are the most likely to tolerate sequence changes. Homology among sequences may be analyzed using, as an example, the BLAST homology searching algorithm of Altschul et al. (ref 12). Alternatively, sequences are modified such that they become more reactive to T- and/or B-cells, based on computer-assisted analysis of probable T- or B-cell epitopes Yet another alternative is to mutate a particular amino acid residue or sequence within the polypeptide in vitro, then screen the mutant polypeptides for their ability to prevent or treat Chlamydia infection according to the method outlined below.

A person skilled in the art will readily understand that by following the screening process of this invention, it will be determined without undue experimentation whether a particular homolog or immunogenic fragment of SEQ ID No. 2, 4, 6 or 8 may be useful in the prevention or treatment of Chlamydia infection. The screening procedure comprises the steps:
(i) immunizing an animal, preferably mouse, with the test homolog or fragment;
(ii) inoculating the immunized animal with infectious Chlamydia; and
(iii) selecting those homologs or fragments which confer protection against Chlamydia.

By “conferring protection” is meant that there is a reduction in severity of any of the effects of Chlamydia infection, in comparison with a control animal which was not immunized with the test homolog or fragment.

Consistent with the first aspect of the invention polypeptide derivatives are provided that are partial nucleic acid sequences of SEQ ID No. 1, 3, 5 or 7, partial sequences of polypeptide sequences hornologousto SEQ ID No. 2, 4, 6 or 8, polypeptides derived from full-length polypeptides by internal deletion, and fusion proteins. It is an accepted practice in the field of immunology to use fragments and variants of protein immunogens as vaccines, as all that is required to induce an immune response to a protein is a small (e.g. 8 to 10 amino acid) immunogenic region of the protein. Various short synthetic peptides corresponding to surface-exposed antigens of pathogens other than Chlamydia have been shown to be effective vaccine antigens against their respective pathogens, e.g. an 11 residue peptide of murine mammary tumor virus (Casey & Davidson, Nucl. Acid Res. (1977) 4:1539), a 16-residue peptide of Semliki Forest virus (Snijders et al., 1991. J. Gen. Virol. 72:55 7-565), and two overlapping peptides of 15 residues each from canine parvovirus (Langeveld et al., Vaccine 12(15):1473-1480, 1994).

Accordingly, it will be readily apparent to one skilled in the art, having read the present description, that partial sequences of SEQ ID No: 2, 4, 6 or 8 or their homologous amino acid sequences are inherent to the full-length sequences and are taught by the present invention. Such polypeptide fragments preferably are at least 12 amino acids in length. Advantageously, they are at least 20 amino acids, preferably at least 50 amino acids, more preferably at least 75 amino acids, and most preferably at least 100 amino acids in length.

Polynucleotides of 30 to 600 nucleotides encoding partial sequences of sequences homologous to SEQ ID No: 2, 4, 6 or 8 are retrieved by PCR amplification using the parameters outlined above and using primers matching the sequences upstream and downstream of the 5′ and 3′ ends of the fragment to be amplified. The template polynucleotide for such amplification is either the full length polynucleotide homologous to SEQ ID No: 1, 3, 5 or 7 or a polynucleotide contained in a mixture of polynucleotides such as a DNA or RNA library. As an alternative method for retrieving the partial sequences, screening hybridization is carried out under conditions described above and using the formula for calculating Tm.

Where fragments of 30 to 600 nucleotides are to be retrieved, the calculated Tm is corrected by subtracting (600/polynucleotide size in base pairs) and the stringency conditions are defined by a hybridization temperature that is 5 to 10° C. below Tm. Where oligonucleotides shorter than 20-30 bases are to be obtained, the formula for calculating the Tm is as follows: Tm=4×(G+C)+2 (A+T). For example, an 18 nucleotide fragment of 50% G+C would have an approximate Tm of 54° C. Short peptides that are fragments of SEQ ID No: 2, 4, 6 or 8 or its homologous sequences, are obtained directly by chemical synthesis.

Epitopes which induce a protective T cell-dependent immune response are present throughout the length of the polypeptide. However, some epitopes may be masked by secondary and tertiary structures of the polypeptide. To reveal such masked epitopes large internal deletions are created which remove much of the original protein structure and exposes the masked epitopes. Such internal deletions sometimes effect the additional advantage of removing immunodominant regions of high variability among strains.

Polynucleotides encoding polypeptide fragments and polypeptides having large internal deletions are constructed using standard methods known in the art. Such methods include standard PCR, inverse PCR, restriction enzyme treatment of cloned DNA molecules. Components for these methods and instructions for their use are readily available from various commercial sources such as Stratagene. Once the deletion mutants have been constructed, they are tested for their ability to prevent or treat Chlamydia infection as described above.

As used herein, a fusion polypeptide is one that contains a polypeptide or a polypeptide derivative of the invention fused at the N- or C-terminal end to any other polypeptide (hereinafter referred to as a peptide tail). A simple way to obtain such a fusion polypeptide is by translation of an in-frame fusion of the polynucleotide sequences, i.e., a hybrid gene. The hybrid gene encoding the fusion polypeptide is inserted into an expression vector which is used to transform or transfect a host cell. Alternatively, the polynucleotide sequence encoding the polypeptide or polypeptide derivative is inserted into an expression vector in which the polynucleotide encoding the peptide tail is already present. Such vectors and instructions for their use are commercially available, e.g. the pMal-c2 or pMal-p2 system from New England Biolabs, in which the peptide tail is a maltose binding protein, the glutathione-S-transferase system of Pharmacia, or the His-Tag system available from Novagen. These and other expression systems provide convenient means for further purification of polypeptides and derivatives of the invention.

An advantageous example of a fusion polypeptide is one where the polypeptide or homolog or fragment of the invention is fused to a polypeptide having adjuvant activity, such as subunit B of either cholera toxin or E. coli heat-labile toxin. Another advantageous fusion is one where the polypeptide, homolog or fragment is fused to a strong T-cell epitope or B-cell epitope. Such an epitope may be one known in the art (e.g. the Hepatitis B virus core antigen, D. R. Millich et al., “Antibody production to the nucleocapsid and envelope of the Hepatitis B virus primed by a single synthetic T cell site”, Nature. 1987. 329:547-549), or one which has been identified in another polypeptide of the invention based on computer-assisted analysis of probable T- or B-cell epitopes. Consistent with this aspect of the invention is a fusion polypeptide comprising T- or B-cell epitopes from SEQ ID No: 2, 4, 6 or 8 or its homolog or fragment, wherein the epitopes are derived from multiple variants of said polypeptide or homolog or fragment, each variant differing from another in the location and sequence of its epitope within the polypeptide. Such a fusion is effective in the prevention and treatment of Chlamydia infection since it optimizes the T- and B-cell response to the overall polypeptide, homolog or fragment.

To effect fusion, the polypeptide of the invention is fused to the N—, or preferably, to the C-terminal end of the polypeptide having adjuvant activity or T- or B-cell epitope. Alternatively, a polypeptide fragment of the invention is inserted internally within the amino acid sequence of the polypeptide having adjuvant activity. The T- or B-cell epitope may also be inserted internally within the amino acid sequence of the polypeptide of the invention.

Consistent with the first aspect, the polynucleotides of the invention also encode hybrid precursor polypeptides containing heterologous signal peptides, which mature into polypeptides of the invention. By “heterologous signal peptide” is meant a signal peptide that is not found in naturally-occurring precursors of polypeptides of the invention.

Polynucleotide molecules according to the invention, including RNA, DNA, or modifications or combinations thereof, have various applications. A DNA molecule is used, for example, (i) in a process for producing the encoded polypeptide in a recombinant host system, (ii) in the construction of vaccine vectors such as poxviruses, which are further used in methods and compositions for preventing and/or treating Chlamydia infection, (iii) as a vaccine agent (as well as an RNA molecule), in a naked form or formulated with a delivery vehicle and, (iv) in the construction of attenuated Chlamydia strains that can over-express a polynucleotide of the invention or express it in a non-toxic, mutated form.

Accordingly, a second aspect of the invention encompasses (i) an expression cassette containing a DNA molecule of the invention placed under the control of or operatively linked to the elements required for expression, also termed an expression control sequence, in particular under the control of an appropriate promoter; (ii) an expression vector containing an expression cassette of the invention; (iii) a procaryotic or eucaryotic cell transformed or transfected with an expression cassette and/or vector of the invention, as well as (iv) a process for producing a polypeptide or polypeptide derivative encoded by a polynucleotide of the invention, which involves culturing a procaryotic or eucaryotic cell transformed or transfected with an expression cassette and/or vector of the invention, under conditions that allow expression of the DNA molecule of the invention and, recovering the encoded polypeptide or polypeptide derivative from the cell culture.

A recombinant expression system is selected from procaryotic and eucaryotic hosts. Eucaryotic hosts include yeast cells (e.g., Saccharomyces cerevisiae or Pichia pastoris), mammalian cells (e.g., COS 1, NIH3T3, or JEG3 cells), arthropods cells (e.g. Spodoptera fruglperda (SF9) cells), and plant cells. A preferred expression system is a procaryotic host such as E. coli. Bacterial and eucaryotic cells are available from a number of different sources including commercial sources to those skilled in the art, e.g., the American Type Culture Collection (ATCC; Rockville, Md.). Commercial sources of cells used for recombinant protein expression also provide instructions for usage of the cells.

The choice of the expression system depends on the features desired for the expressed polypeptide. For example, it may be useful to produce a polypeptide of the invention in a particular lipidated form or any other form.

One skilled in the art would readily understand that not all vectors and expression control sequences and hosts would be expected to express equally well the polynucleotides of this invention. With the guidelines described below, however, a selection of vectors, expression control sequences and hosts may be made without undue experimentation and without departing from the scope of this invention.

In selecting a vector, the host must be chosen that is compatible with the vector which is to exist and possibly replicate in it. Considerations are made with respect to the vector copy number, the ability to control the copy number, expression of other proteins such as antibiotic resistance. In selecting an expression control sequence, a number of variables are considered. Among the important variable are the relative strength of the sequence (e.g. the ability to drive expression under various conditions), the ability to control the sequence's function, compatibility between the polynucleotide to be expressed and the control sequence (e.g. secondary structures are considered to avoid hairpin structures which prevent efficient transcription). In selecting the host, unicellular hosts are selected which are compatible with the selected vector, tolerant of any possible toxic effects of the expressed product, able to secrete the expressed product efficiently if such is desired, to be able to express the product in the desired conformation, to be easily scaled up, and to which ease of purification of the final product.

The choice of the expression cassette depends on the host system selected as well as the features desired for the expressed polypeptide. Typically, an expression cassette includes a promoter that is functional in the selected host system and can be constitutive or inducible; a ribosome binding site; a start codon (ATG) if necessary; a region encoding a signal peptide, e.g., a lipidation signal peptide; a DNA molecule of the invention; a stop codon; and optionally a 3′ terminal region (translation and/or transcription terminator). The signal peptide encoding region is adjacent to the polynucleotide of the invention and placed in proper reading frame. The signal peptide-encoding region is homologous or heterologous to the DNA molecule encoding the mature polypeptide and is compatible with the secretion apparatus of the host used for expression. The open reading frame constituted by the DNA molecule of the invention, solely or together with the signal peptide, is placed under the control of the promoter so that transcription and translation occur in the host system. Promoters and signal peptide encoding regions are widely known and available to those skilled in the art and include, for example, the promoter of Salmonella typhimurium (and derivatives) that is inducible by arabinose (promoter araB) and is functional in Gram-negative bacteria such as E. coli (as described in U.S. Pat. No. 5,028,530); the promoter of the gene of bacteriophage T7 encoding RNA polymerase, that is functional in a number of E. coli strains expressing T7 polymerase (described in U.S. Pat. No. 4,952,496); OspA lipidation signal peptide; and RlpB lipidation signal peptide (Takase et al., J. Bact. (1987) 169:5692).

The expression cassette is typically part of an expression vector, which is selected for its ability to replicate in the chosen expression system. Expression vectors (e.g. plasmids or viral vectors) can be chosen, for example, from those described in Pouwels et al. (Cloning Vectors: A Laboratory Manual 1985, Supp. 1987). Suitable expression vectors can be purchased from various commercial sources.

Methods for transforming/transfecting host cells with expression vectors are well-known in the art and depend on the host system selected.

Upon expression, a recombinant polypeptide of the invention (or a polypeptide derivative) is produced and remains in the intracellular compartment, is secreted/excreted in the extracellular medium or in the periplasmic space, or is embedded in the cellular membrane. The polypeptide is recovered in a substantially purified form from the cell extract or from the supernatant after centrifugation of the recombinant cell culture. Typically, the recombinant polypeptide is purified by antibody-based affinity purification or by other well-known methods that can be readily adapted by a person skilled in the art, such as fusion of the polynucleotide encoding the polypeptide or its derivative to a small affinity binding domain. Antibodies useful for purifying by immunoaffinity the polypeptides of the invention are obtained as described below.

A polynucleotide of the invention can also be useful as a vaccine. There are two major routes, either using a delivery vehicle viral or bacterial or synthetic (ie live vaccine vector or microparticles) or administering the gene in a free form, e.g., inserted into a nucleic acid vector. Therapeutic or prophylactic efficacy of a polynucleotide of the invention is evaluated as described below.

Accordingly, a further aspect of the invention provides (i) a vaccine vector such as a poxvirus, containing a DNA molecule of the invention, placed under the control of elements required for expression; (ii) a composition of matter comprising a vaccine vector of the invention, together with a diluent or carrier; specifically (iii) a pharmaceutical composition containing a therapeutically or prophylactically effective amount of a vaccine vector of the invention; (iv) a method for inducing an immune response against Chlamydia in a mammal (e.g., a human; alternatively, the method can be used in veterinary applications for treating or preventing Chlamydia infection of animals, e.g. cats or birds), which involves administering to the mammal an immunogenically effective amount of a vaccine vector of the invention to elicit a protective or therapeutic immune response to Chlamydia; and particularly, (y) a method for preventing and/or treating a Chlamydia (e.g., C. trachomatis, C. psittaci, C. pneumonia, C. pecorum) infection, which involves administering a prophylactic or therapeutic amount of a vaccine vector of the invention to an infected individual.

Additionally, a further aspect of the invention encompasses the use of a vaccine vector of the invention in the preparation of a medicament for preventing and/or treating Chlamydia infection.

As used herein, a vaccine vector expresses one or several polypeptides or derivatives of the invention. The vaccine vector may express additionally a cytokine, such as interleukin-2 (IL-2) or interleukin-12 (IL-12), that enhances the immune response (adjuvant effect). It is understood that each of the components to be expressed is placed under the control of elements required for expression in a mammalian cell.

Consistent with a further aspect of the invention is a composition comprising several vaccine vectors, each of them capable of expressing a polypeptide or derivative of the invention. A composition may also comprise a vaccine vector capable of expressing an additional Chlamydia antigen, or a subunit, fragment, homolog, mutant, or derivative thereof optionally together with or a cytokine such as IL-2 or IL-12.

Vaccination methods for treating or preventing infection in a mammal comprises use of a vaccine vector of the invention to be administered by any conventional route, particularly to a mucosal (e.g., ocular, intranasal, oral, gastric, pulmonary, intestinal, rectal, vaginal, or urinary tract) surface or via the parenteral (e.g., subcutaneous, intradermal, intramuscular, intravenous, or intraperitoneal) route. Preferred routes depend upon the choice of the vaccine vector. Treatment may be effected in a single dose or repeated at intervals. The appropriate dosage depends on various parameters understood by skilled artisans such as the vaccine vector itself, the route of administration or the condition of the mammal to be vaccinated (weight, age and the like).

Live vaccine vectors available in the art include viral vectors such as adenoviruses, poxviruses and alphavirus, as well as bacterial vectors, e.g. Shigella, Salmonella, Vibrio cholerae, Lactobacillus, Bacille bilié de Calmette-Guérin (BCG), and Streptococcus.

An example of an adenovirus vector, as well as a method for constructing an adenovirus vector capable of expressing a DNA molecule of the invention, are described in U.S. Pat. No. 4,920,209. Poxvirus vectors include vaccinia and canary pox virus, described in U.S. Pat. No. 4,722,848 and U.S. Pat. No. 5,364,773, respectively. For a description of a vaccinia virus vector (canary pox) see Taylor et al, (ref 13). The canarypox vectors have limited or no replication in mammalian cells.

Generally, the dose of vaccine viral vector, for therapeutic or prophylactic use, can be of from about 1×10⁴ to about 1×10¹¹, advantageously from about 1×10⁷ to about 1×10¹⁰, preferably of from about 1×10⁷ to about 1×10⁹ plaque-forming units per kilogram. Preferably, viral vectors are administered parenterally; for example, in 3 doses, 4 weeks apart. It is preferable to avoid adding a chemical adjuvant to a composition containing a viral vector of the invention and thereby minimizing the immune response to the viral vector itself.

Alphavirus vectors may include Simliki Forest virus vectors (ref 16), Sindbis virus vectors (ref 17) or Venezuelan Equine Encephalitis virus vectors (ref 18). Naked RNA or plasmid DNA can be used efficiently for immunization as well as recombinant particles which may contain replication defective alphaviruses.

Non-toxicogenic Vibrio cholerae mutant strains that are useful as a live oral vaccine are known. U.S. Pat. No. 4,882,278, describe strains which have a substantial amount of the coding sequence of each of the two ctxA alleles deleted so that no functional cholerae toxin is produced. An effective vaccine dose of a Vibrio cholerae strain capable of expressing a polypeptide or polypeptide derivative encoded by a DNA molecule of the invention contains about 1×10⁵ to about 1×10⁹, preferably about 1×10⁶ to about 1×10⁸, viable bacteria in a volume appropriate for the selected route of administration. Preferred routes of administration include all mucosal routes; most preferably, these vectors are administered intranasally or orally.

Attenuated Salmonella typhimurium strains, genetically engineered for recombinant expression of heterologous antigens or not, and their use as oral vaccines are described in U.S. Pat. No. 5,851,519 issued Dec. 22, 1998. Preferred routes of administration include all mucosal routes; most preferably, these vectors are administered intranasally or orally.

Other attenuated bacterial strains used as vaccine vectors in the context of the present invention are described in U.S. Pat. No. 5,643,771 issued Jul. 1, 1997.

In bacterial vectors, the polynucleotide of the invention is inserted into the bacterial genome or remains in a free state as part of a plasmid. The bacterial vectors can be used to express the chlamydia vaccine antigen or deliver to the host cell an expression vector such as plasmid DNA which is subsequently expressed in the host cell and elicits an immune response to the chlamydial antigen.

The composition comprising a vaccine bacterial vector of the present invention may further contain an adjuvant. A number of adjuvants are known to those skilled in the art. Preferred adjuvants include, but are not limited to aluminum salts (alum), such as aluminum hydroxide, aluminum phosphate, aluminum sulfate, oil-in water emulsion formulations, saponin adjuvants such as ISCOMs, cytokines such as interleukins, interferons, macrophage colony stimulating factor, tumor necrosis factor.

Vaccines or immunogenic compositions according to the invention may be either prophylactic (i.e. to prevent disease) or therapeutic (i.e. to treat disease after infection). Immunogenic compositions used as vaccines comprise an immunologically effective amount of the antigen or immunogenic fragment of the antigen. By immunologically effective amount it is meant that the administration of that amount to an individual, either as a single dose or as part of a series of doses, is effective for the prevention or treatment. The term therapeutically effect amount refers to an amount of a therapeutic agent to treat ameliorate, or prevent a desired disease or condition, or to exhibit a detectable therapeutic or preventative effect. For the purposes of the present invention, an effective dose will be from 1 μg/kg to 100 μg/kg or 10 μg/kg to 50 μg/kg.

Immunogenic compositions and vaccines may be administered parentally, by injection subcutaneous, intradermal or intramuscularly injection. Alternatively, the immunogenic compositions formulated according to the present invention, may be formulated and delivered in a manner to evoke an immune response at mucosal surfaces. Thus, the immunogenic composition may be administered to mucosal surfaces by, for example, the nasal or oral (intagastric) routes. Alternatively, other modes of administration including suppositories and oral formulations may be desirable. For suppositories, binders and carriers may include, for example, polyalkalene glycols or triglycerides, Such suppositories may be formed from mixtures containing the active immunogenic ingredient(s) in the range of about 10%, preferably about 1 to 2%. Oral formulations may include normally employed carriers, such as, pharmaceutical grades of saccharine, cellulose and magnesium carbonate. These compositions can take the form of solutions, suspensions, tablets, pills, capsules, sustained release formulations or powders and contain about 1 to 95% of the active ingredients, preferably about 20 to 75%.

Accordingly, an additional aspect of the invention provides (i) a composition of matter comprising a polynucleotide of the invention, together with a diluent or carrier; (ii) a pharmaceutical composition comprising a therapeutically or prophylactically effective amount of a polynucleotide of the invention; (iii) a method for inducing an immune response against Chlamydia in a mammal by administration of an immunogenically effective amount of a polynucleotide of the invention to elicit a protective immune response to Chlamydia; and particularly, (iv) a method for preventing and/or treating a Chlamydia (e.g. C. trachomatis, C. psittaci, C. pneumoniae, or C. pecorum) infection, by administering a prophylactic or therapeutic amount of a polynucleotide of the invention to an infected individual. Additionally, the fourth aspect of the invention encompasses the use of a polynucleotide of the invention in the preparation of a medicament for preventing and/or treating Chlamydia infection. A preferred use includes the use of a DNA molecule placed under conditions for expression in a mammalian cell, especially in a plasmid that is unable to replicate in mammalian cells and to substantially integrate in a mammalian genome.

Use of the polynucleotides of the invention include their administration to a mammal as a vaccine, for therapeutic or prophylactic purposes. Such polynucleotides are used in the form of DNA as part of a plasmid that is unable to replicate in a mammalian cell and unable to integrate into the mammalian genome. Typically, such a DNA molecule is placed under the control of a promoter suitable for expression in a mammalian cell. The promoter functions either ubiquitously or tissue-specifically. Examples of non-tissue specific promoters include the early Cytomegalovirus (CMV) promoter (described in U.S. Pat. No. 4,168,062) and the Rous Sarcoma Virus promoter (described in Norton & Coffin, Molec. Cell Biol. (1985) 5:28 1). An example of a tissue specific promoter is the desmin promoter which drives expression in muscle cells (Li & Paulin, J. Biol. Chem. (1993) 268:10403). Use of promoters is well-known to those skilled in the art. Useful vectors are described in numerous publications, specifically WO 94/21797.

Polynucleotides of the invention which are used as vaccines encode either a precursor or a mature form of the corresponding polypeptide. In the precursor form, the signal peptide can be either homologous or heterologous. In the latter case, a eucaryotic leader sequence can be used.

As used herein, a composition of the invention contains one or several polynucleotides with optionally at least one additional polynucleotide encoding another Chlamydia antigen, or a fragment, derivative, mutant, or analog thereof. The composition may also contain an additional polynucleotide encoding a cytokine, such as interleukin-2 (IL-2) or interleukin-12 (IL-12) so that the immune response is enhanced. These additional polynucleotides are placed under appropriate control for expression. Advantageously, DNA molecules of the invention and/or additional DNA molecules to be included in the same composition, are present in the same plasmid.

Standard techniques of molecular biology for preparing and purifying polynucleotides are used in the preparation of polynucleotide therapeutics of the invention. For use as a vaccine, a polynucleotide of the invention is formulated according to various methods outlined below.

One method utilizes the polynucleotide in a naked form, free of any delivery vehicles. Such a polynucleotide is simply diluted in a physiologically acceptable solution such as sterile saline or sterile buffered saline, with or without a carrier. When present, the carrier preferably is isotonic, hypotonic, or weakly hypertonic, and has a relatively low ionic strength, such as provided by a sucrose solution, e.g., a solution containing 20% sucrose.

An alternative method utilizes the polynucleotide in association with agents that assist in cellular uptake. Examples of such agents are (i) chemicals that modify cellular permeability, such as bupivacaine (see, e.g., WO 94/16737), (ii) liposomes for encapsulation of the polynucleotide, or (iii) cationic lipids or silica, gold, or tungsten microparticles which associate themselves with the polynucleotides.

Anionic and neutral liposomes are well-known in the art (see, e.g. Liposomes: A Practical Approach, RPC New Ed, IRL press (1990), for a detailed description of methods for making liposomes) and are useful for delivering a large range of products, including polynucleotides.

Cationic lipids are also known in the art and are commonly used for gene delivery. Such lipids include Lipofectin™ also known as DOTMA (N-[1-(2,3-dioleyloxy)propyl]-N,N,N-trimethylammonium chloride), DOTAP (1,2-bis(oleyloxy)-3-(trimethylammonio)propane), DDAB (dimethyldioctadecylammonium bromide), DOGS (dioctadecylamidologlycyl spermine) and cholesterol derivatives such as DC-Chol (3 beta-(N—(N˜N′-dimethyl aminomethane)-carbamoyl) cholesterol). A description of these cationic lipids can be found in EP 187,702, WO 90/11092, U.S. Pat. No. 5,283,185, WO 91/1 5501, WO 95/26356, and U.S. Pat. No. 5,527,928. Cationic lipids for gene delivery are preferably used in association with a neutral lipid such as DOPE (dioleyl phosphatidylethanolamine), as described in WO 90/11092 as an example.

Formulation containing cationic liposomes may optionally contain other transfection-facilitating compounds.

Gold or tungsten microparticles are used for gene delivery, as described in WO 91/00359, WO 93/1 7706, and Tang et al. (ref 19). The microparticlecoated polynucleotide is injected via intradermal or intraepidermal routes using a needleless injection device (“gene gun”), such as those described in U.S. Pat. No. 4,945,050, U.S. Pat. No. 5,015,580, and WO 94/24263.

The amount of DNA to be used in a vaccine recipient depends, e.g. on the strength of the promoter used in the DNA construct, the immunogenicity of the expressed gene product, the condition of the mammal intended for administration (e.g., the weight, age, and general health of the mammal), the mode of administration, and the type of formulation. In general, a therapeutically or prophylactically effective dose from about 1 μg to about 1 mg, preferably, from about 10 μg to about 800 μg and, more preferably, from about 25 μg to about 250 μg, can be administered to human adults. The administration can be achieved in a single dose or repeated at intervals.

The route of administration is any conventional route used in the vaccine field. As general guidance, a polynucleotide of the invention is administered via a mucosal surface, e.g., an ocular, intranasal, pulmonary, oral, intestinal, rectal, vaginal, and urinary tract surface; or via a parenteral route, e.g., by an intravenous, subcutaneous, intraperitoneal, intradermal, intraepidermal, or intramuscular route. The choice of administration route depends on the formulation that is selected. A polynucleotide formulated in association with bupivacaine is advantageously administered into muscles. When a neutral or anionic liposome or a cationic lipid, such as DOTMA or DC-Chol, is used, the formulation can be advantageously injected via intravenous, intranasal (aerosolization), intramuscular, intradermal, and subcutaneous routes. A polynucleotide in a naked form can advantageously be administered via the intramuscular, intradermal, or sub-cutaneous routes.

Although not absolutely required, such a composition can also contain an adjuvant. If so, a systemic adjuvant that does not require concomitant administration in order to exhibit an adjuvant effect is preferable such as, e.g., QS21, which is described in U.S. Pat. No. 5,057,546.

The sequence information provided in the present application enables the design of specific nucleotide probes and primers that are used for diagnostic purposes. Accordingly, a fifth aspect of the invention provides a nucleotide probe or primer having a sequence found in or derived by degeneracy of the genetic code from a sequence shown in SEQ ID No: 1 or 3.

The term “probe” as used in the present application refers to DNA (preferably single stranded) or RNA molecules (or modifications or combinations thereof) that hybridize under the stringent conditions, as defined above, to nucleic acid molecules having SEQ ID No: 1 or to sequences homologous to SEQ ID No: 1 or 3, or to its complementary or anti-sense sequence. Generally, probes are significantly shorter than full-length sequences. Such probes contain from about 5 to about 100, preferably from about 10 to about 80, nucleotides. In particular, probes have sequences that are at least 75%, preferably at least 85%, more preferably 95% homologous to a portion of SEQ ID No: 1 or that are complementary to such sequences. Probes may contain modified bases such as inosine, methyl-5-deoxycytidine, deoxyuridine, dimethylamino-5-deoxyuridine, or diamino-2,6-purine. Sugar or phosphate residues may also be modified or substituted. For example, a deoxyribose residue may be replaced by a polyamide and phosphate residues may be replaced by ester groups such as diphosphate, alkyl, arylphosphonate and phosphorothioate esters. In addition, the 2′-hydroxyl group on ribonucleotides may be modified by including such groups as alkyl groups.

Probes of the invention are used in diagnostic tests, as capture or detection probes. Such capture probes are conventionally immobilized on a solid support, directly or indirectly, by covalent means or by passive adsorption. A detection probe is labelled by a detection marker selected from: radioactive isotopes, enzymes such as peroxidase, alkaline phosphatase, and enzymes able to hydrolyze a chromogenic, fluorogenic, or luminescent substrate, compounds that are chromogenic, fluorogenic, or luminescent, nucleotide base analogs, and biotin.

Probes of the invention are used in any conventional hybridization technique, such as dot blot, Southern blot (Southern, J. Mol. Biol. (1975) 98:503), northern blot (identical to Southern blot with the exception that RNA is used as a target), or the sandwich technique (Dunn et al., Cell (1977) 12:23). The latter technique involves the use of a specific capture probe and/or a specific detection probe with nucleotide sequences that at least partially differ from each other.

A primer is a probe of usually about 10 to about 40 nucleotides that is used to initiate enzymatic polymerization of DNA in an amplification process (e.g. PCR), in an elongation process, or in a reverse transcription method. Primers used in diagnostic methods involving PCR are labeled by methods known in the art.

As described herein, the invention also encompasses (i) a reagent comprising a probe of the invention for detecting and/or identifying the presence of Chlamydia in a biological material; (ii) a method for detecting and/or identifying the presence of Chlamydia in a biological material, in which (a) a sample is recovered or derived from the biological material, (b) DNA or RNA is extracted from the material and denatured, and (c) exposed to a probe of the invention, for example, a capture, detection probe or both, under stringent hybridization conditions, such that hybridization is detected; and (iii) a method for detecting and/or identifying the presence of Chlamydia in a biological material, in which (a) a sample is recovered or derived from the biological material, (b) DNA is extracted therefrom, (c) the extracted DNA is primed with at least one, and preferably two, primers of the invention and amplified by polymerase chain reaction, and (d) the amplified DNA fragment is produced.

It is apparent that disclosure of polynucleotide sequences of SEQ ID No: 1, 3, 5 or 7, its homologs and partial sequences enable their corresponding amino acid sequences. Accordingly, a sixth aspect of the invention features a substantially purified polypeptide or polypeptide derivative having an amino acid sequence encoded by a polynucleotide of the invention.

A “substantially purified polypeptide” as used herein is defined as a polypeptide that is separated from the environment in which it naturally occurs and/or that is free of the majority of the polypeptides that are present in the environment in which it was synthesized. For example, a substantially purified polypeptide is free from cytoplasmic polypeptides. Those skilled in the art would readily understand that the polypeptides of the invention may be purified from a natural source, i.e., a Chlamydia strain, or produced by recombinant means.

Consistent with the sixth aspect of the invention are polypeptides, homologs or fragments which are modified or treated to enhance their immunogenicity in the target animal, in whom the polypeptide, homolog or fragments are intended to confer protection against Chlamydia. Such modifications or treatments include: amino acid substitutions with an amino acid derivative such as 3-methylhistidine, 4-hydroxyproline, 5-hydroxylysine etc., modifications or deletions which are carried out after preparation of the polypeptide, homolog or fragment, such as the modification of free amino, carboxyl or hydroxyl side groups of the amino acids.

Identification of homologous polypeptides or polypeptide derivatives encoded by polynucleotides of the invention which have specific antigenicity is achieved by screening for cross-reactivity with an antiserum raised against the polypeptide of reference having an amino acid sequence of SEQ ID No: 1, 3, 5 or 7. The procedure is as follows:

a monospecific hyperimmune antiserum is raised against a purified reference polypeptide, a fusion polypeptide (for example, an expression product of MBP, GST, or His-tag systems, the description and instructions for use of which are contained in Invitrogen product manuals for pcDNA3.1/Myc-His(+) A, B, and C and for the Xpress™ System Protein Purification), or a synthetic peptide predicted to be antigenic. Where an antiserum is raised against a fusion polypeptide, two different fusion systems are employed. Specific antigenicity can be determined according to a number of methods, including Western blot, dot blot, and ELISA, as described below.

In a Western blot assay, the product to be screened, either as a purified preparation or a total E. coli extract, is submitted to SDS-Page electrophoresis as described by Laemmli (Nature (1970) 227:680). After transfer to a nitrocellulose membrane, the material is further incubated with the monospecific hyperimmune antiserum diluted in the range of dilutions from about 1:5 to about 1:5000, preferably from about 1:100 to about 1:500. Specific antigenicity is shown once a band corresponding to the product exhibits reactivity at any of the dilutions in the above range.

In an ELISA assay, the product to be screened is preferably used as the coating antigen. A purified preparation is preferred, although a whole cell extract can also be used. Briefly, about 100 μl of a preparation at about 10 μg protein/ml are distributed into wells of a 96-well polycarbonate ELISA plate. The plate is incubated for 2 hours at 37° C. then overnight at 4° C. The plate is washed with phosphate buffer saline (PBS) containing 0.05% Tween 20 (PBS/Tween buffer). The wells are saturated with 250 μl PBS containing 1% bovine serum albumin (BSA) to prevent non-specific antibody binding. After 1 hour incubation at 37° C., the plate is washed with PBS/Tween buffer. The antiserum is serially diluted in PBS/Tween buffer containing 0.5% BSA. 100 μl of dilutions are added per well. The plate is incubated for 90 minutes at 37° C., washed and evaluated according to standard procedures. For example, a goat anti-rabbit peroxidase conjugate is added to the wells when specific antibodies were raised in rabbits. Incubation is carried out for 90 minutes at 37° C. and the plate is washed. The reaction is developed with the appropriate substrate and the reaction is measured by colorimetry (absorbance measured spectrophotometrically). Under the above experimental conditions, a positive reaction is shown by O.D. values greater than a non immune control serum.

In a dot blot assay, a purified product is preferred, although a whole cell extract can also be used. Briefly, a solution of the product at about 100 μg/ml is serially twofold diluted in 50 mM Tris-HCl (pH 7.5). 100 μl of each dilution are applied to a nitrocellulose membrane 0.45 μm set in a 96-well dot blot apparatus (Biorad). The buffer is removed by applying vacuum to the system. Wells are washed by addition of 50 mM Tris-HCl (pH 7.5) and the membrane is air-dried. The membrane is saturated in blocking buffer (50 mM Tris-HCl (pH 7.5) 0.15 M NaCl, 10 g/L skim milk) and incubated with an antiserum dilution from about 1:50 to about 1:5000, preferably about 1:500. The reaction is revealed according to standard procedures. For example, a goat anti-rabbit peroxidase conjugate is added to the wells when rabbit antibodies are used. Incubation is carried out 90 minutes at 37° C. and the blot is washed. The reaction is developed with the appropriate substrate and stopped. The reaction is measured visually by the appearance of a colored spot, e.g., by colorimetry. Under the above experimental conditions, a positive reaction is shown once a colored spot is associated with a dilution of at least about 1:5, preferably of at least about 1:500.

Therapeutic or prophylactic efficacy of a polypeptide or derivative of the invention can be evaluated as described below. A seventh aspect of the invention provides (i) a composition of matter comprising a polypeptide of the invention together with a diluent or carrier; specifically (ii) a pharmaceutical composition containing a therapeutically or prophylactically effective amount of a polypeptide of the invention; (iii) a method for inducing an immune response against Chlamydia in a mammal, by administering to the mammal an immunogenically effective amount of a polypeptide of the invention to elicit a protective immune response to Chlamydia; and particularly, (iv) a method for preventing and/or treating a Chlamydia (e.g., C. trachomatis. C. psittaci, C. pneumoniae, or C. pecorum) infection, by administering a prophylactic or therapeutic amount of a polypeptide of the invention to an infected individual. Additionally, the seventh aspect of the invention encompasses the use of a polypeptide of the invention in the preparation of a medicament for preventing and/or treating Chlamydia infection.

As used herein, the immunogenic compositions of the invention are administered by conventional routes known the vaccine field, in particular to a mucosal (e.g., ocular, intranasal, pulmonary, oral, gastric, intestinal, rectal, vaginal, or urinary tract) surface or via the parenteral (e.g., subcutaneous, intradermal, intramuscular, intravenous, or intraperitoneal) route. The choice of administration route depends upon a number of parameters, such as the adjuvant associated with the polypeptide. If a mucosal adjuvant is used, the intranasal or oral route is preferred. If a lipid formulation or an aluminum compound is used, the parenteral route is preferred with the sub-cutaneous or intramuscular route being most preferred. The choice also depends upon the nature of the vaccine agent. For example, a polypeptide of the invention fused to CTB or LTB is best administered to a mucosal surface.

As used herein, the composition of the invention contains one or several polypeptides or derivatives of the invention. The composition optionally contains at least one additional Chlamydia antigen, or a subunit, fragment, homolog, mutant, or derivative thereof.

For use in a composition of the invention, a polypeptide or derivative thereof is formulated into or with liposomes, preferably neutral or anionic liposomes, microspheres, ISCOMS, virus-like-particles (VLPs) or bacterial ghosts (EP 1 158 966B1) to facilitate delivery and/or enhance the immune response. These compounds are readily available to one skilled in the art.

Treatment is achieved in a single dose or repeated as necessary at intervals, as can be determined readily by one skilled in the art. For example, a priming dose is followed by three booster doses at weekly or monthly intervals. An appropriate dose depends on various parameters including the recipient (e.g., adult or infant), the particular vaccine antigen, the route and frequency of administration, the presence/absence or type of adjuvant, and the desired effect (e.g., protection and/or treatment), as can be determined by one skilled in the art. In general, a vaccine antigen of the invention is administered by a mucosal route in an amount from about 10 μg to about 500 μg, preferably from about 1 μg to about 200 μg. For the parenteral route of administration, the dose usually does not exceed about 1 mg, preferably about 100 μg.

When used as vaccine agents, polynucleotides and polypeptides of the invention may be used sequentially as part of a multistep immunization process. For example, a mammal is initially primed with a vaccine vector of the invention such as a pox virus, e.g., via the parenteral route, and then boosted twice with the polypeptide encoded by the vaccine vector, e.g., via the mucosal route. In another example, liposomes associated with a polypeptide or derivative of the invention is also used for priming, with boosting being carried out mucosally using a soluble polypeptide or derivative of the invention in combination with a mucosal adjuvant (e.g., LT).

A polypeptide derivative of the invention is also used in accordance with the seventh aspect as a diagnostic reagent for detecting the presence of anti-Chlamydia antibodies, e.g. in a blood sample. Such polypeptides are about 5 to about 80, preferably about 10 to about 50 amino acids in length. They are either labeled or unlabeled, depending upon the diagnostic method. Diagnostic methods involving such a reagent are described below.

Upon expression of a DNA molecule of the invention, a polypeptide or polypeptide derivative is produced and purified using known laboratory techniques. As described above, the polypeptide or polypeptide derivative may be produced as a fusion protein containing a fused tail that facilitates purification. The fusion product is used to immunize a small mammal, e.g., a mouse or a rabbit, in order to raise antibodies against the polypeptide or polypeptide derivative (monospecific antibodies). Accordingly, an eighth aspect of the invention provides a monospecific antibody that binds to a polypeptide or polypeptide derivative of the invention.

By “monospecific antibody” is meant an antibody that is capable of reacting with a unique naturally-occurring Chlamydia polypeptide. An antibody of the invention is either polyclonal or monoclonal. Monospecific antibodies may be recombinant, e.g., chimeric (e.g., constituted by a variable region of murine origin associated with a human constant region), humanized (a human immunoglobulin constant backbone together with hypervariable region of animal, e.g., murine, origin), and/or single chain. Both polyclonal and monospecific antibodies may also be in the form of immunoglobulin fragments, e.g., F(ab)2 or Fab fragments. The antibodies of the invention are of any isotype, e.g., IgG or IgA, and polyclonal antibodies are of a single isotype or a mixture of isotypes.

Antibodies against the polypeptides, homologs or fragments of the present invention are generated by immunization of a mammal with a composition comprising said polypeptide, homolog or fragment. Such antibodies may be polyclonal or monoclonal. Methods to produce polyclonal or monoclonal antibodies are well known in the art.

The antibodies of the invention, which are raised to a polypeptide or polypeptide derivative of the invention, are produced and identified using standard immunological assays, e.g., Western blot analysis, dot blot assay, or ELISA. The antibodies are used in diagnostic methods to detect the presence of a Chlamydia antigen in a sample, such as a biological sample. The antibodies are also used in affinity chromatography for purifying a polypeptide or polypeptide derivative of the invention. As is discussed further below, such antibodies may be used in prophylactic and therapeutic passive immunization methods.

Accordingly, a further aspect of the invention provides (i) a reagent for detecting the presence of Chlamydia in a biological sample that contains an antibody, polypeptide, or polypeptide derivative of the invention; and (ii) a diagnostic method for detecting the presence of Chlamydia in a biological sample, by contacting the biological sample with an antibody, a polypeptide, or a polypeptide derivative of the invention, such that an immune complex is formed, and by detecting such complex to indicate the presence of Chlamydia in the sample or the organism from which the sample is derived.

Those skilled in the art will readily understand that the immune complex is formed between a component of the sample and the antibody, polypeptide, or polypeptide derivative, whichever is used, and that any unbound material is removed prior to detecting the complex. It is understood that a polypeptide reagent is useful for detecting the presence of anti-Chlamydia antibodies in a sample, e.g., a blood sample, while an antibody of the invention is used for screening a sample, such as a gastric extract or biopsy, for the presence of Chlamydia polypeptides.

For diagnostic applications, the reagent (i.e., the antibody, polypeptide, or polypeptide derivative of the invention) is either in a free state or immobilized on a solid support, such as a tube, a bead, or any other conventional support used in the field. Immobilization is achieved using direct or indirect means. Direct means include passive adsorption (non-covalent binding) or covalent binding between the support and the reagent. By “indirect means” is meant that an anti-reagent compound that interacts with a reagent is first attached to the solid support. For example, if a polypeptide reagent is used, an antibody that binds to it can serve as an anti-reagent, provided that it binds to an epitope that is not involved in the recognition of antibodies in biological samples. Indirect means may also employ a ligand-receptor system, for example, where a molecule such as a vitamin is grafted onto the polypeptide reagent and the corresponding receptor immobilized on the solid phase. This is illustrated by the biotin-streptavidin system. Alternatively, a peptide tail is added chemically or by genetic engineering to the reagent and the grafted or fused product immobilized by passive adsorption or covalent linkage of the peptide tail.

Such diagnostic agents may be included in a kit which also comprises instructions for use. The reagent is labeled with a detection means which allows for the detection of the reagent when it is bound to its target. The detection means may be a fluorescent agent such as fluorescein isocyanate or fluorescein isothiocyanate, or an enzyme such as horseradish peroxidase or luciferase or alkaline phosphatase, or a radioactive element such as ¹²⁵I or ⁵¹Cr.

Accordingly, another aspect of the invention provides a process for purifying, from a biological sample, a polypeptide or polypeptide derivative of the invention, which involves carrying out antibody-based affinity chromatography with the biological sample, wherein the antibody is a monospecific antibody of the invention.

For use in a purification process of the invention, the antibody is either polyclonal or monospecific, and preferably is of the IgG type. Purified IgGs is prepared from an antiserum using standard methods. Conventional chromatography supports, as well as standard methods for grafting antibodies, are described in, e.g., Antibodies: A Laboratory Manual, D. Lane, E. Harlow, Eds. (1988) and outlined below.

Briefly, a biological sample, such as an C. trachomatis extract preferably in a buffer solution, is applied to a chromatography material, preferably equilibrated with the buffer used to dilute the biological sample so that the polypeptide or polypeptide derivative of the invention (i.e., the antigen) is allowed to adsorb onto the material. The chromatography material, such as a gel or a resin coupled to an antibody of the invention, is in either a batch form or a column. The unbound components are washed off and the antigen is then eluted with an appropriate elution buffer, such as a glycine buffer or a buffer containing a chaotropic agent, e.g., guanidine HCl, or high salt concentration (e.g., 3 M MgCl₂). Eluted fractions are recovered and the presence of the antigen is detected, e.g., by measuring the absorbance at 280 nm.

A further aspect of the invention provides (i) a composition of matter comprising a monospecific antibody of the invention, together with a diluent or carrier; (ii) a pharmaceutical composition comprising a therapeutically or prophylactically effective amount of a monospecific antibody of the invention, and (iii) a method for treating or preventing a Chlamydia (e.g. C. trachomatis, C. psittaci, C. pneumoniae or C. pecorum) infection, by administering a therapeutic or prophylactic amount of a monospecific antibody of the invention to an infected individual. Additionally, the eleventh aspect of the invention encompasses the use of a monospecific antibody of the invention in the preparation of a medicament for treating or preventing Chlamydia infection.

The monospecific antibody is either polyclonal or monoclonal, preferably of the IgA isotype (predominantly). In passive immunization, the antibody is administered to a mucosal surface of a mammal, e.g., the gastric mucosa, e.g., orally or intragastrically, advantageously, in the presence of a bicarbonate buffer. Alternatively, systemic administration, not requiring a bicarbonate buffer, is carried out. A monospecific antibody of the invention is administered as a single active component or as a mixture with at least one monospecific antibody specific for a different Chlamydia polypeptide. The amount of antibody and the particular regimen used are readily determined by one skilled in the art. For example, daily administration of about 100 to 1,000 μg of antibodies over one week.

Therapeutic or prophylactic efficacy are evaluated using standard methods in the art, e.g., by measuring induction of a mucosal immune response or induction of protective and/or therapeutic immunity, using, e.g., chlamydia mouse model disclosed herein. Those skilled in the art will readily recognize that the strain of chlamydia used in the model may be replaced with another Chlamydia strain or serovar. For example, the efficacy of DNA molecules and polypeptides from C. trachomatis is preferably evaluated in a mouse model using C. trachomatis strain. Protection is determined by comparing the degree of Chlamydia infection to that of a control group. Protection is shown when infection is reduced by comparison to the control group. Statistical analysis may be employed to demonstrate differences from the control group. Such an evaluation is made for polynucleotides, vaccine vectors, polypeptides and derivatives thereof, as well as antibodies of the invention.

Adjuvants useful in any of the vaccine compositions described above are as follows.

Adjuvants for parenteral administration include aluminum compounds, such as aluminum hydroxide, aluminum phosphate, and aluminum hydroxy phosphate. The antigen is precipitated with, or adsorbed onto, the aluminum compound according to standard protocols. Other adjuvants, such as RIBI (ImmunoChem, Hamilton, Mont.), are used in parenteral administration.

Adjuvants for mucosal administration include bacterial toxins, e.g., the cholera toxin (CT), the E. coli heat-labile toxin (LT), the Clostridium difficile toxin A and the pertussis toxin (PT), or combinations, subunits, toxoids, or mutants thereof such as a purified preparation of native cholera toxin subunit B (CTB). Fragments, homologs, derivatives, and fusions to any of these toxins are also suitable, provided that they retain adjuvant activity. Preferably, a mutant having reduced toxicity is used. Other adjuvants, such as a bacterial monophosphoryl lipid A (MPLA) of, e.g., E. coli, Salmonella minnesota, Salmonella typhimurium, or Shigella flexneri; saponins, or polylactide glycolide (PLGA) microspheres, is also be used in mucosal administration.

Adjuvants useful for both mucosal and parenteral administrations include polyphosphazene (WO 95/02415), DC-chol (3 b-(N—(N′,N′-dimethyl aminomethane)carbamoyl) cholesterol; U.S. Pat. No. 5,283,185 and WO 96/14831) and QS-21 (WO 88/09336).

Any pharmaceutical composition of the invention containing a polynucleotide, a polypeptide, a polypeptide derivative, or an antibody of the invention, is manufactured in a conventional manner. In particular, it is formulated with a pharmaceutically acceptable diluent or carrier, e.g., water or a saline solution such as phosphate buffer saline. In general, a diluent or carrier is selected on the basis of the mode and route of administration, and standard pharmaceutical practice.

The data presented herein and described in detail below demonstrates that nucleic acid immunization with the Chlamydia nucleic acid molecule encoding Mgp002 gene elicits immune responses and produces significant protective immunity to lung challenge infection with C. trachomatis MoPn.

It is clearly apparent to one skilled in the art, that the various embodiments of the present invention have many applications in the fields of vaccination, diagnosis and treatment of chlamydial infections. A further non-limiting discussion of such uses is further presented below.

### EXAMPLES

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### Example 1

This Example illustrates the preparation of a plasmid vector for immunization.

The C. trachomatis mouse pneumonitis (MoPn) isolate was grown in HeLa 229 cells in Eagle MEM containing 10% fetal bovine serum and 2 mM L-glutamine. The MoPn EBs were harvested and purified by step gradient density centrifugation at 43,000 g for 60 min at 4° C. The purified EBs were washed twice with PBS, centrifugated at 30,000 g for 30 min, resuspended in sucrose-phosphate-glutamic acid (SPG) buffer and frozen at −70° C. until used.

The nucleic acid molecule encoding Mgp002 gene was cloned into eukaryotic expression plasmid pCAMycHis inframe with the Myc-His tags present in the vector. This vector was constructed from pcDNA3.1(−)Myc-His C (Invitrogen, San Diego) and plasmid VR1012 (Vical). The details of the construction are disclosed in the PCT publication WO 00/55326 published on Sep. 21, 2000. Briefly, plasmid pcDNA3.1(−)Myc-His C (Invitrogen) was restricted with Spe I and Bam HI to remove the CMV promoter and the remaining vector fragment was isolated. The CMV promoter and intron A from plasmid VR-1012 (Vical) was isolated on a Spe I/Bam HI fragment. The fragments were ligated together to produce plasmid pCA/Myc-His.

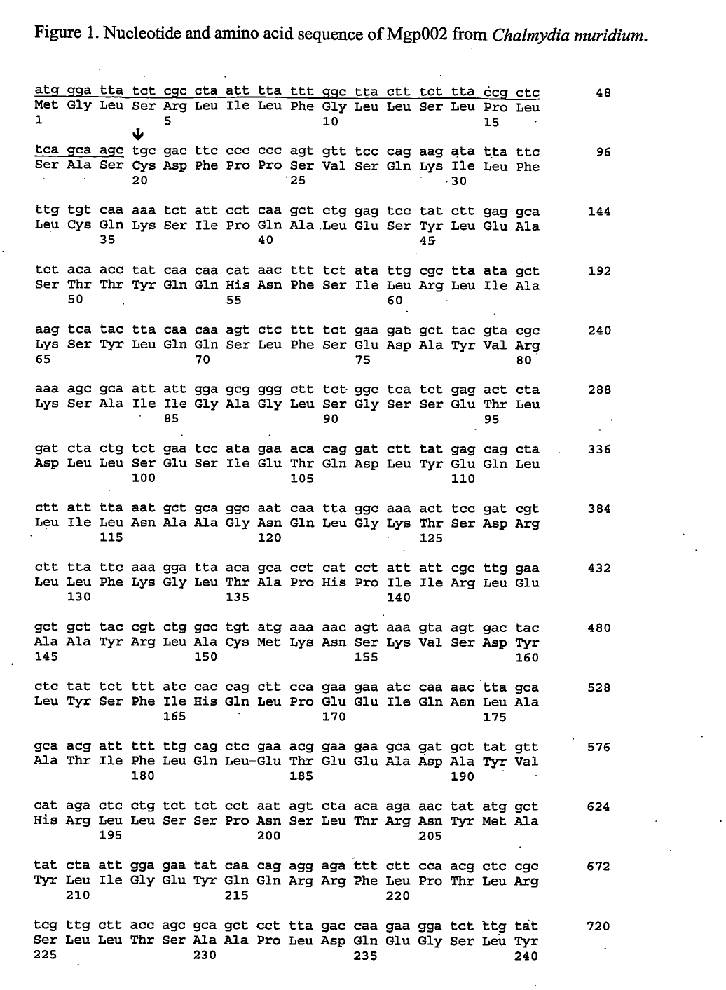

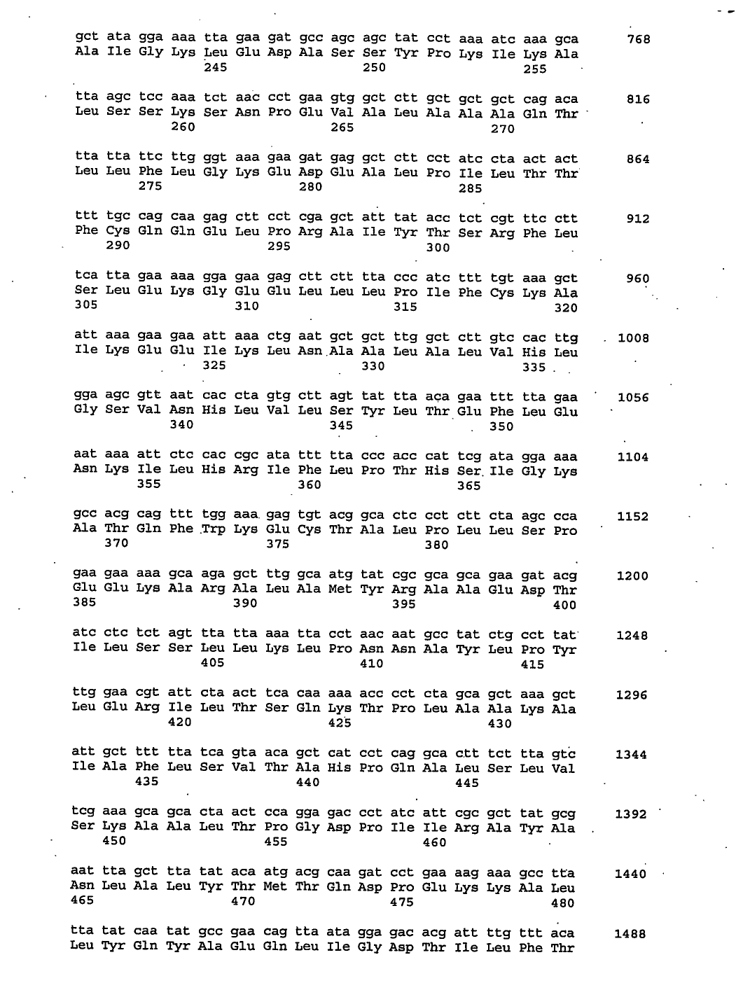

The full-length mgp002 gene was amplified from MoPn genomic DNA by polymerase chain reaction (PCR) with a 5′ primer (5′ ATAAGAATGCGGCCGCCACC ATG GGA TTA TCT CGC CTA ATT 3′-SEQ ID No: 9) which included a NotI site (underlined), a start codon (bold), and the N-terminal sequence of the mature Mgp002 gene product of MoPn and a 3′ reverse primer (5′ GTTGGTACCGAGCTCGCTCCACTATTCTCATTAATAATCC 3′-SEQ ID No: 10) which include a Kpn I site (underlined). The reverse primer is complementary to the 3′end of the Mgp002 gene, but does not contain a stop codon. Instead, an additional nucleotide was inserted, leading to an in-frame gene fusion with the Myc- and His-tags of pCAMycHis. The PCR product was isolated after agarose gel electrophoresis, restricted with Kpn I and NotI and ligated into the Kpn I and NotI sites of vector pCAMycHis. The ligation mixture was transformed into E. coli DH10b under ampicillin selection. In order to verify the correct amplification and cloning, the DNA of the entire insert was sequenced. The resulting plasmid was named pCACTMgp002. The PCR product, had the nucleic acid sequence shown in FIG. 1 (SEQ ID No: 1) and the deduced amino acid sequence (SEQ ID No: 2) which represented the full-length Mgp002 gene.

The signal sequence deleted mgp002gene was also amplified from MoPn genomic DNA by polymerase chain reaction (PCR) with a forward primer 5′ ATAAGAATGCGGCCGCCACCATGTGCGACTTCCCCCCCAGT 3′-SEQ ID No:11 and mgp002 reverse primer 5′ GTTGGTACCGAGCTCGCTCCACTATTCTCATTAATAATCC 3′ SEQ ID No:12, as described above. The resulting plasmid, cloned into pCAMycHis was identified as pCACTMgp002delta. The deleted putative signal sequence is shown in FIG. 1 as underlined and the signal sequence deleted Mgp002 gene had the nucleic acid sequence indicated to start at the arrow in FIG. 1 (SEQ ID No:5) and the deduced amino acid sequence (SEQ ID No:6).

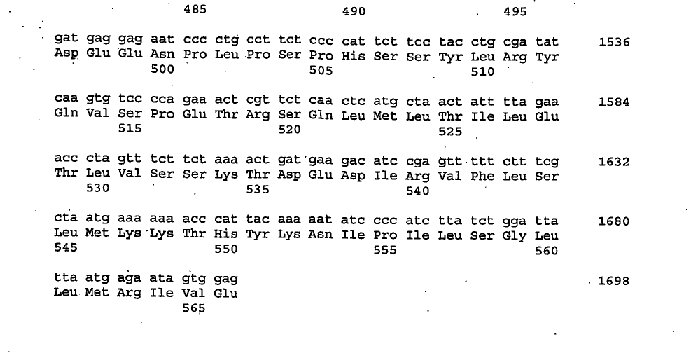

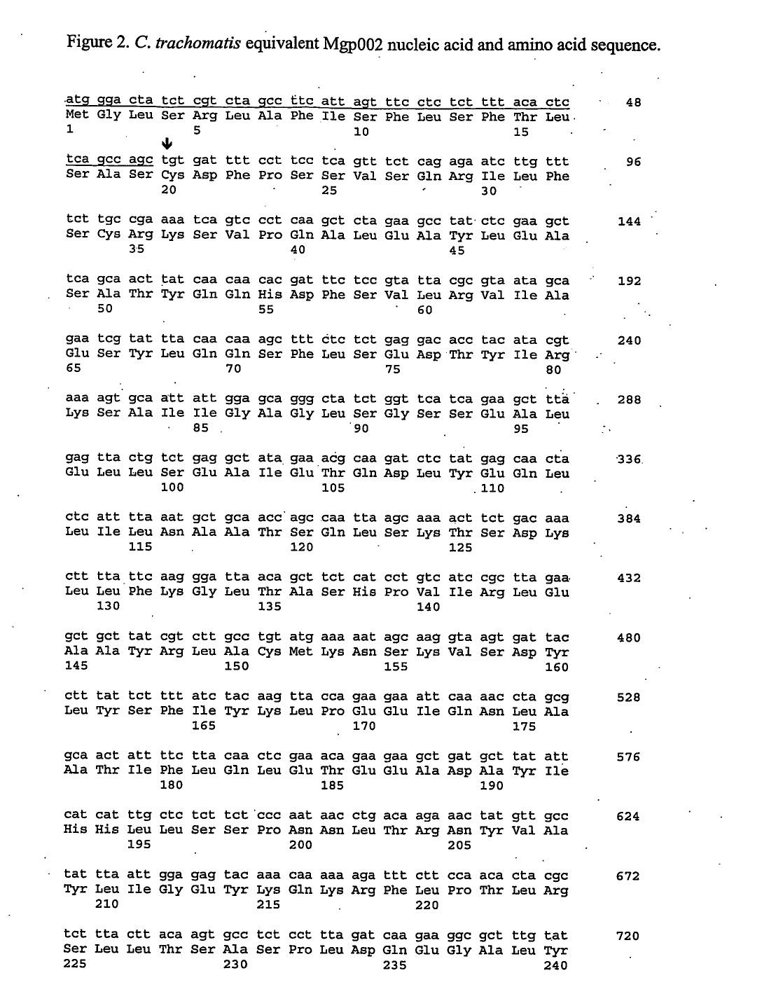

Similarly, the Mgp002 gene, from the Chlamydia trachomatis serovar D nucleic acid sequence shown in FIG. 2 (SEQ ID No:3) and deduced protein sequence (SEQ ID No:4) for the full-length Mgp002 gene, or the signal sequence deleted gene shown in FIG. 2 at the arrow for the nucleic acid sequence (SEQ ID No:7) and deduced protein sequence (SEQ ID No:8). One skilled in the art can appreciate that any other sequence from any other serovar, can be obtained using similar techniques as outlined above.

### Example 2

This Example shows the results of immunizing studies using the nucleic acid vector.

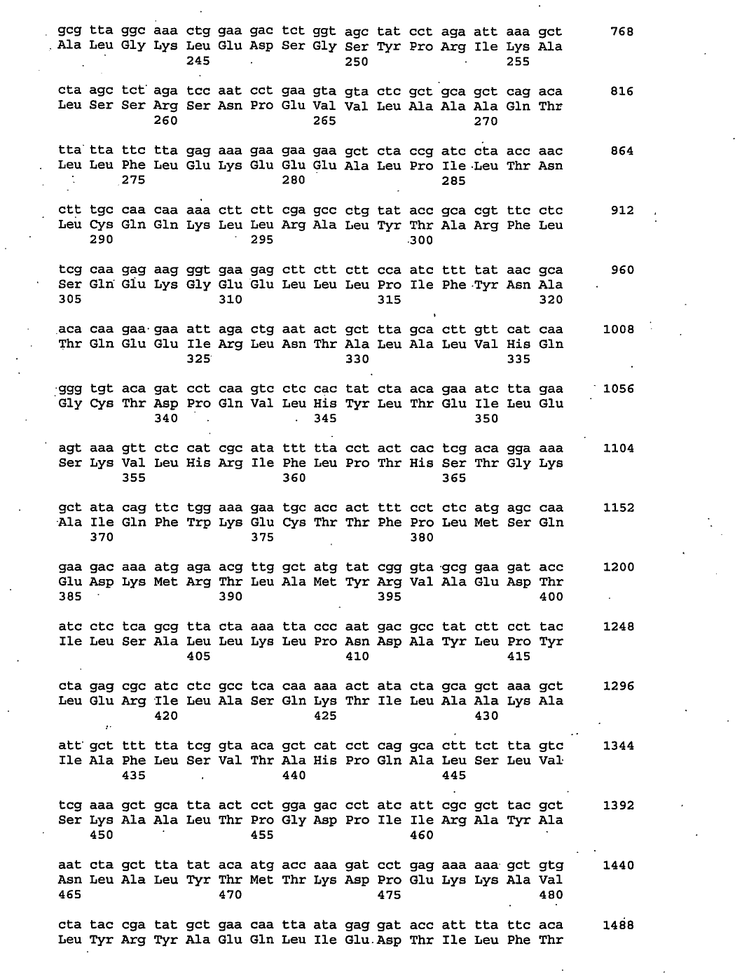

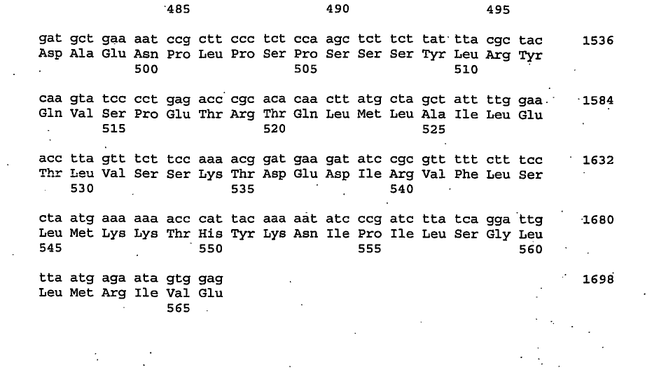

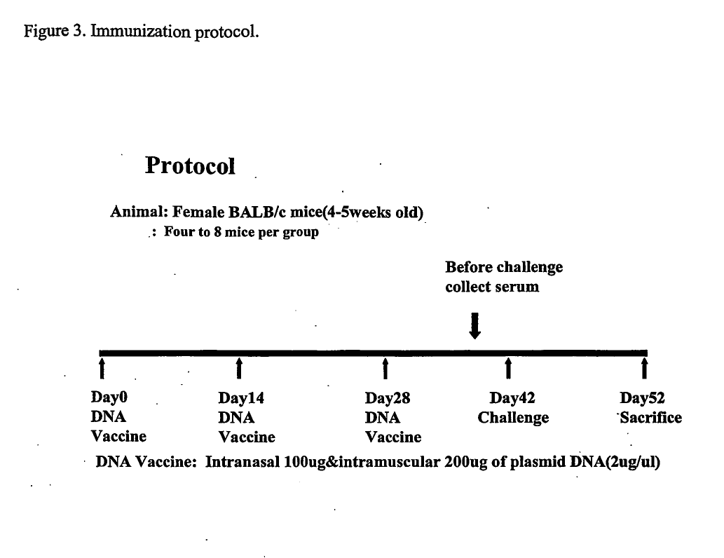

In order to investigate whether the immune responses elicited by the nucleic acid immunization were functionally significant, in vivo protective efficacy was evaluated as described before (ref 20). Briefly, female Balb/c mice (4 to 5 weeks old) were purchased from Charles River Canada (St. Constant, Canada) mice were intramuscularly and intranasally immunized with plasmid DNA, prepared as described in Example 1, on three occasions, at 0, 2 and 4 weeks see FIG. 3. For each immunization, a total of 200 μg DNA in 200 μl was injected into the two quadriceps muscles (100 μg of DNA/injection site) using a 27-gauge needle. At the same time, 50 μg DNA in 50 μl was delivered onto the nostrils of mice with a micropipette. The droplet was subsequently inhaled by the mice.

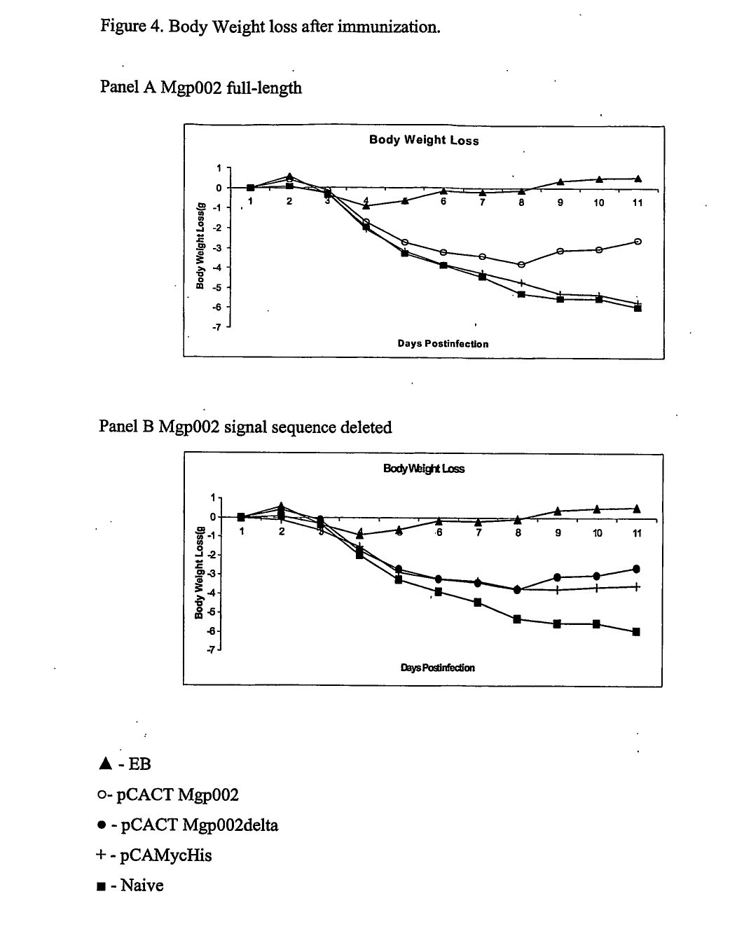

Mice were challenged intranasally with 2×10³ IFU of C. trachomatis MoPn EB 14 days after last immunization, as described. Briefly, after ether anesthesia 25 μl of SPG containing an inoculum of 2×10³ IFU of MoPn was delivered onto the nostrils of mice with a micropipette. The droplet was subsequently inhaled by the mice. Body weight was measured daily for 10 days following the challenge infection as a measure of chlamydia-induced morbidity see FIG. 4. Mice injected with saline (naïve) or with the blank vector (pCAMycHis) were used as negative controls. After postinfection day 3, mice immunized with Mgp002 gene product or the truncated form, lost significantly less body mass than did the negative control group (FIG. 4).

On postinfection day 10, the mice were sacrificed and their lungs were aseptically isolated and homogenized with grinder in SPG buffer. The tissue suspensions were centrifuged at 500 g for 10 min at 4° C. remove coarse tissue and debris. Supernatants, were frozen at −70° C. until tissue culture testing for quantitative growth of the organism.

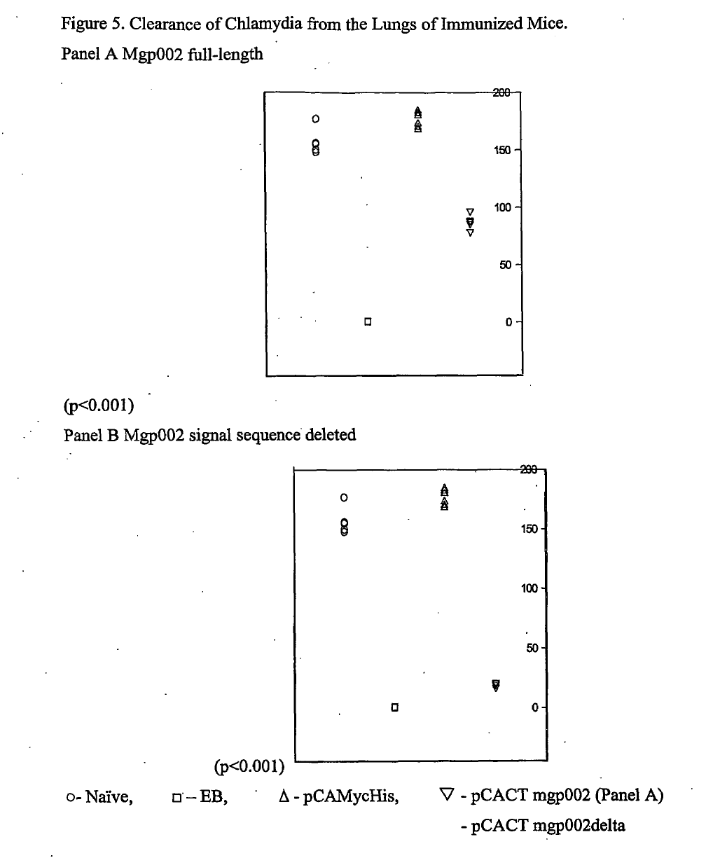

For more direct measure of the effectiveness of the DNA vaccination, the ability to limit the in vivo growth of Chlamydia following a sublethal lung infection was evaluated. In this infection model system, postchallenge day 10 is the time of peak growth and was chosen for comparison of lung titers among the various groups of mice. Mice immunized with the Mgp002 full-length gene product DNA had a lung titer (IFU per 200× field) significantly lower (p<0.001) than negative control groups (pCAMycHis alone and naïve saline groups) as shown in FIG. 5. Surprisingly the mice immunized with the truncated form of the Mgp002 gene (FIG. 5 Panel B) showed even lower IFUs than the full-length gene.

These data demonstrate that nucleic acid immunization with the Mgp002 and even the truncated form of the gene elicits protective immune responses to lung challenge infection with C. trachomatis MoPn. These data also demonstrate that the protective sequences in the Mgp002 gene reside in the truncated form of the gene.

### Example 3

This example illustrates the preparation of a nucleic acid vector for recombinant mgp002 expression in E. coli.

Procedures required for PCR amplification, DNA modifications by endo- and exonucleases for generating desired ends for cloning of DNA, ligation, and bacterial transformation are well known in the art. Standard molecular cloning techniques used there are well known in the art and are described by Sambrook, J., Fritsch, E. F. and Maniatis, T. Molecular Cloning: A Laboratory Manual, 2^{nd} ed.; Cold Spring Harbor Laboratory: Cold Spring Harbo, N.Y. and by Ausubel et al., Current Protocols in Molecular Biology, Greene Publishing and Wiley-Interscience; 1987.

Chlamydia genomic DNA was prepared from Chlamydia trachomatis mouse pneumonitis strain (MoPn, also known as Chlamydia muridarum) after passage of bacteria in McCoy cells.

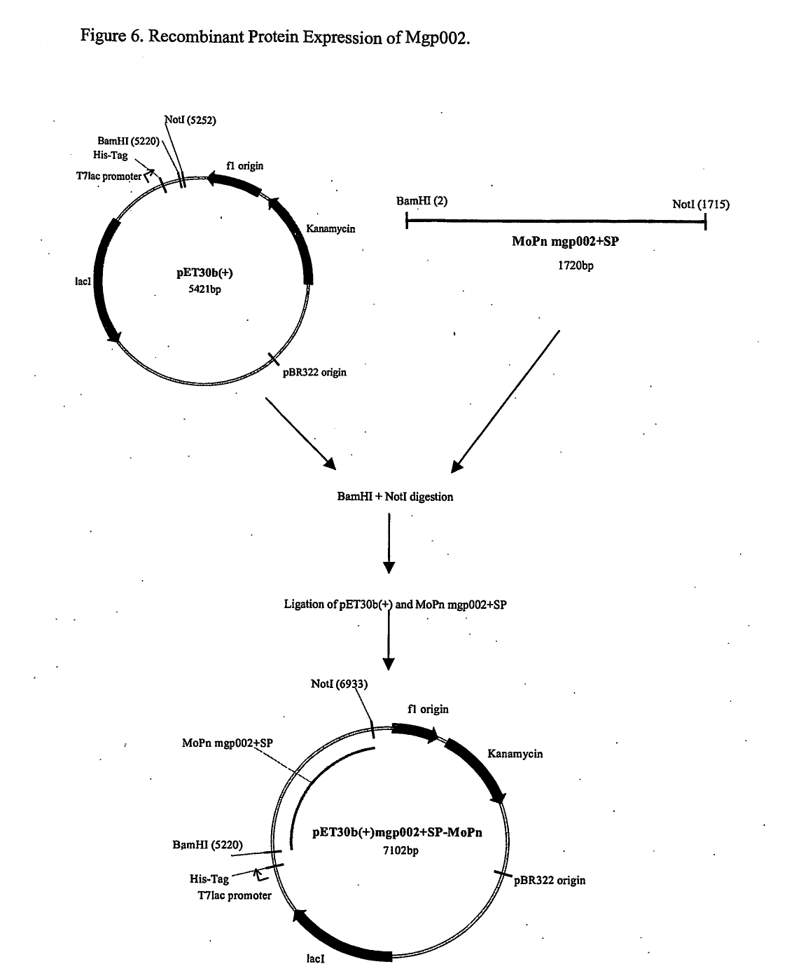

For expression, mgp002 coding sequence with its native signal peptide (encoded by first 18 codons) was amplified from total DNA harvested from C. trachomatis MoPn infected McCoy cells using forward primer MoPn mgp002−F/+SP (5′-GAATTCGGATCCGATGGGATTATCTCGCCTA-3′) SEQ ID No:13, and reverse primer MoPn mgp002-R (5′-ATTAAGAATGCGGCCGCTTTATCACTCCACTATTCT-3′) SEQ ID No:14 and Advantage-HF2 Polymerase Mix (Clontech). The forward primer introduced sequence encoding a BamHI restriction site (italics). The reverse primer introduced a NotI restriction site (italics) and a double-stop codon (underlined on the complimentary strand). The resulting PCR product was restricted sequentially with BamHI and NotI and inserted into the pET30b(+) plasmid, which had also been cut with BamHI and NotI. The new plasmid was designated pET30b(+)mgp002+SP. In this construct, mgp002+SP is expressed with an N-terminal His-Tag®, originating from an upstream coding sequence within the pET30b(+) vector. FIG. 6 illustrates a graphical representation of the cloning steps described above. Similar procedures can be utilized for the preparation of Mgp002 from Chlamydia trachomatis serovar D or any other serovar strain. The amino acid sequence has the same sequence as illustrated in FIG. 1 (SEQ ID No:2) except for the addition of the N-terminal His tag to facilitate purification.

For expression of recombinant mgp002 protein, an over night culture (85 ml) of E. coli BL21(DE3) harbouring expression vector pET30b(+)mgp002+SP#1 was used to inoculate flasks containing 500 ml of Luria-Bertani broth each at 37° C. until A₅₉₅ of 0.8 was attained. Expression of mgp002 as a His-tagged protein was induced by addition of IPTG at a final concentration of 1 mM, and the culture was incubated for an additional 4 h. Over-expressed recombinant protein was then analysed on Coomassie-Blue-stained SDS-PAGE and by immuno-staining with and Anti-His-tag monoclonal antibody to verify expression using standard conditions.

### Example 4

This example illustrates the purification of His-tagged recombinant Mgp002 protein from E. coli using immobilized metal affinity chromatography (IMAC).

The bacterial cell culture expressing the recombinant Mgp002 from Example 3 were centrifuged to pellet the cells and mixed with phosphate buffered saline (PBS; 10 mM phosphate buffer, pH 7.5, 150 mM NaCl) containing 0.5% v/v Triton X-100, at a ratio of approximately 1 g wet wt/mL (typically 20-30 g/30 mL). Tubes containing the mixture were chilled on ice and sonicated with a Branson Sonifier at 20-30% power output for three one minute intervals, with intervening cooling periods of 1-2 minutes. The resultant solution was transferred to 40 mL Beckman centrifuge tubes and centrifuged on a Beckman Avanti J30i centrifuge at 10,000 rpm for 15 minutes at 4 C. The supernatant was decanted, and the centrifuged pellet was resuspended in an equal volume of the same buffer containing 6 M guanidine hydrochloride. The mixture was sonicated and centrifuged as described, and the supernatant, containing the solubilized mgp002 protein, was retained as the feed material.

The column used for the IMAC purification was the Amersham XK 50/20 type, with a 2.5 cm radius. It was packed with Amersham Pharmacia chelating Separose Fast Flow to a height of 10 cm, for a column volume (CV) of 200 mL. If previously used, the column was regenerated and sanitized according to the manufacturer's instructions; following the passage of 7 CV of deionized water, the column was charged with 1 CV of 0.1 M NiCl₂, and equilibrated with 4 CV PBS, pH 6.8.

The column was equilibrated with 4 CV of the guanidine containing buffer described above, at a flow rate of 25 mL/min. 500 mL of sample feed was loaded at 25 mL/min., followed by a 3 CV wash step with PBS containing 50 mM imidazole. Elution of the mgp002 protein was effected by running through the column 3 CV of PBS containing 300 mM imidazole. The eluate fraction was retained for diafiltration.

Finally, The eluate was concentrated by approximately 6-fold with a Pall Minum tangential Flow filtration device, using a 10 kDa nominal molecular weight cut-off filter. To ensure solubility of the product, the concentrate was diafiltered in the same apparatus with approximately ten volumes of buffer containing 10 mM Tris-HCl, pH 8.5, 150 mM NaCl, 0.8 M L-arginine, and 10 mM dithiothreitol. This resulted in a purified recombinant Mgp002 protein suitable for formulating into an immunogenic composition or vaccine with or without an adjuvant.

### Example 5

This example illustrates the protection from genital challenge with Chlamydia trachomatis Serovar D in immunized CH3 mice.

The purified recombinant Mgp002 protein (20 ug/dose) from Example 4 was formulated with an ISCOM adjuvant ISCOMATRIX (IMX) dose of 2.5 ug/immunization. Protection was measured by determining the bacterial load in genital washes following intravaginal challenge with Serovar D Chlamydia trachomatis.

Briefly, CH3 female mice were immunized with each of the test antigens in IMX two times. Animals were then be induced into an estrous-like state using progesterone (depo provera) and then challenged intravaginally with Chlamydia trachomatis serovar D. Washes and swabs were taken at time points following infection and evaluated in culture for inclusion forming units (IFU). A positive culture from any time point indicated that the animal in question was considered infected. Five time points were be evaluated to determine what level of infection occurred. The immunization protocol is shown in the following Table 1.

TABLE 1
Immunization Protocol
Animal species: C3H mice

| | |
|---|---|
| Day 0 | Immunize with the various protein combination in ISCOMATRIX ™ |
| Day 7 | Administer depo provera to group A. 2.5 mg in 200 ul s.c. |
| Day 14 | Pre-swab mice of group A by rotating calcium alginate swab 4-5 times in vaginally |
| | cavity. Challenge mice with indicated dose of C. Trachomatis in 10 μl. Make sure the |
| | mice remain motionless on their backs for at least 1 hour and the inoculum remains |
| | in the vaginal cavity during that time. |
| Day 14 | Immunize with the various protein combination in ISCOMATRIX ™ |
| Day 28 | Administer depo provera to group A to H. 2.5 mg in 200 ul s.c. |
| Day 34 | Bleed all groups |
| Day 35 | Pre-swab mice of all groups by rotating calcium alginate swab 4-5 times in vaginally |
| | cavity. Challenge mice with indicated dose of C. Trachomatis in 10 μl. The mice |
| | immobilized on their backs for at least 1 hour and the inoculum remains in the |
| | vaginal cavity during that time. |
| Day 38 | Monitor; wash with 2 × 50 μl SPG, and swab by rotating swab 4-5 times in the |
| | vaginal cavity. |
| Day 40 | Monitor; wash with 2 × 50 μl SPG, and swab by rotating swab 4-5 times in the |
| | vaginal cavity. |
| Day 42 | Monitor; wash with 2 × 50 μl SPG, and swab by rotating swab 4-5 times in the |
| | vaginal cavity. |
| Day 46 | Monitor; wash with 2 × 50 μl SPG, and swab by rotating swab 4-5 times in the |
| | vaginal cavity. |
| Day 48 | Monitor; wash with 2 × 50 μl SPG, and swab by rotating swab 4-5 times in the |
| | vaginal cavity. |

On Days 3, 5, 7, 11 and 14 the vaginally cavity was washed with 2×50 μl SPG buffer followed by a swab. The washes and swab were added to a tube containing 400 μl SPG and placed on ice where they were either frozen for later testing or tested immediately. On Day 34, the mice from all groups were bleed and the serum samples sent to Ausra Raudonikiene/Kiristin Boehlke (Bld 17, rm 124), where the samples will be spun down and the serum removed and frozen until testing.

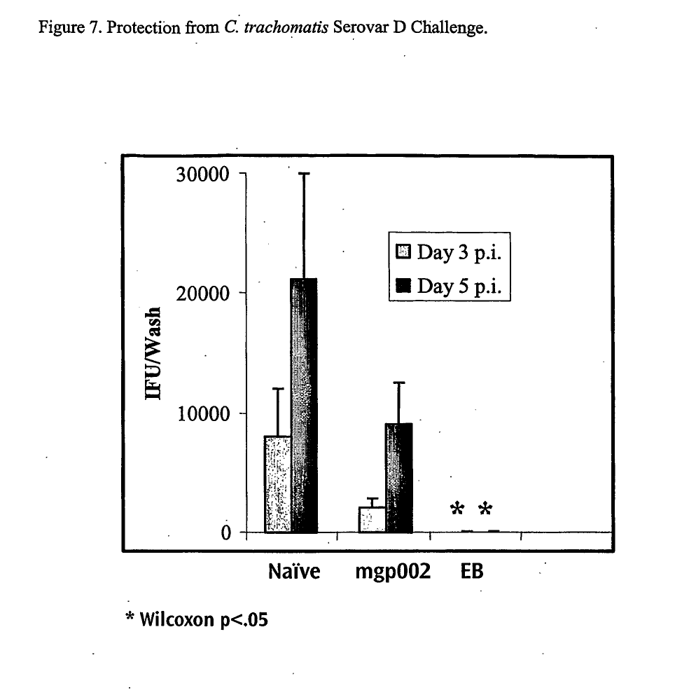

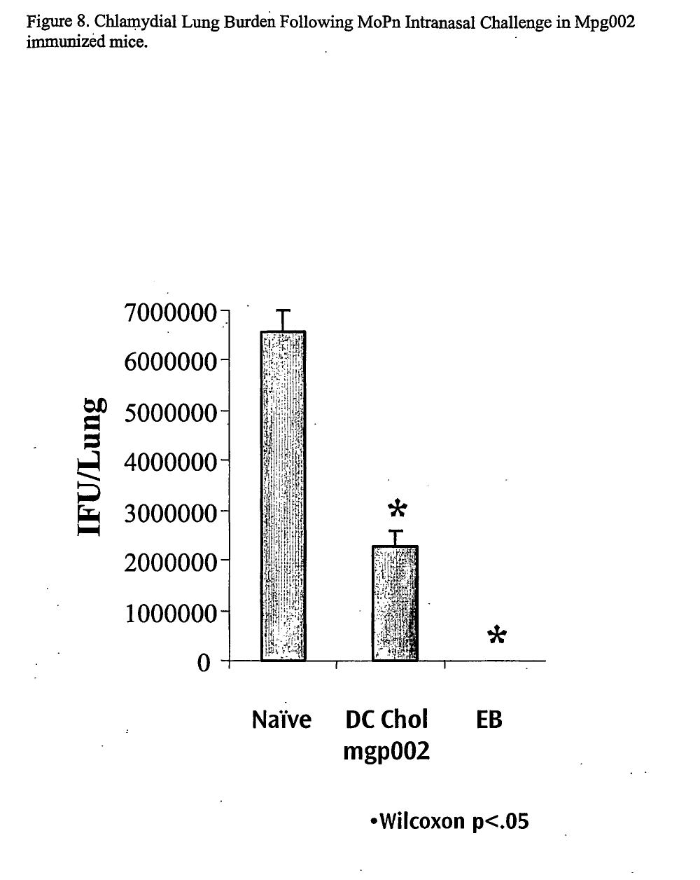

FIG. 7 shows that Mgp002 protein immunization was able to drastically reducing the bacterial burden in the genital tract at the day 3 time period and less so for at day 5. These results demonstrate that recombinant forms of mgp002 are able to provide protection through reductions in bacterial load following challenge. Elementary bodies (EB) were a positive control and also able to reduce bacterial burden in the genital tract. These results were statistically significant (Wilcoxon p<0.05) when compared to the control groups which only got adjuvant and placebo.

### Example 6

This example illustrates the protection from a lung challenge with Chlamydia trichomatis MoPn in Mgp002 immunized Balb/c mice.

The lung challenge was performed as describe above in Example 2. The Mgp002 protein used to immunize the mice was the same as described in Example 4. Briefly, mice were immunized three times intramuscularly (i.m) (see FIG. 3) with the purified recombinant Mgp002 protein (25 ug/dose) from Example 4 formulated with DC-Chol adjuvant dose of 200 ug/immunization. Mice were challenged intranasally (i.n) with 2×10³ IFU of C. trachomatis MoPn EB 14 days after last immunization, as described in Example 2.

On postinfection day 10, the mice were sacrificed and their lungs were aseptically isolated and homogenized with grinder in SPG buffer. The tissue suspensions were centrifuged at 500 g for 10 min at 4° C. remove coarse tissue and debris. Supernatants, were frozen at −70° C. until tissue culture testing for quantitative growth of the organism. FIG. 8 demonstrated that mice immunized with Mgp002 recombinant protein formulated with another adjuvant, DC-Chol, also showed significant reduction in chlamydial burden in the lungs when compared with the unimmunized mice. These results were statistically significant at p<0.05.

### SUMMARY OF DISCLOSURE

In summary of this disclosure, the present invention provides a method of nucleic acid, including DNA, immunization of a host, including humans, against disease caused by infection by strain of Chlamydia, specifically C. trachomatis, employing a nucleic acid vector, specifically a plasmid vector, containing a nucleotide sequence encoding a full-length or a truncated form of the Mgp002 gene product of a strain of Chlamydia and a promoter to effect expression of Mgp002 gene and the truncated form in the host. Both the full-length and the truncated form of the Mgp002 gene elicited a protective immune response in the host, against challenge from live chlamydia. The truncated form elicited an even greater protective response than the full-length form. Modifications are possible within the scope of this invention.

### REFERENCES

1. M. A. Liu, M. R. Hilleman, R. Kurth, Ann. N.Y. Acad. Sci. 772 (1995).
2. D. M. Pardoll and A. M. Beckerieg, Immunity 3, 165 (1995).
3. W. M. McDonnell and F. K. Askari, N. Engl. J. Med. 334, 42 (1996).
4. J. B. Ulmer of al., Science 259, 1745 (1993).
5. B. Wang et al., Proc. Natl. Acad. Sci. USA 90, 4156 (1993).
6. Schachter J. In: Chlamydia: Intracellular Biology, Pathogenesis and Immunology, Stephens R (Ed) 139-169 (1999).
7. G. J. M. Cox, T. J. Zamnb, L. A. Babiuk, J. Virol. 67, 5664 (1993).
8. Z. Q. Xiang et al., Virology 199, 132 (1994).
9. Igietseme J U and Murdin A. Infect Immun 68:6798-6806 (2000).
10. J. J. Donnelly et al., J. Infect. Dis. 713, 314 (1996).
11. H. D. Caldwell and Judd R. C. Infect Immun 38:960-968 (1982)
12. Altschul et al., Nucleic Acids Res. 25:3389-3402 (1997)
13. Taylor et al, Vaccine 13:539 (1995)
14. Stephens R S, et al., Science 282:754-759 (1998).
15. Read T D et al., Nucleic Acids Res. 28:1397-1406 (2000).
16. Liljestrom P, Garoff H. Biotechnology 9(12):1356-61 (1991).
17. Dubensky T W et al. J. Virol. 70(1):508-19 (1996).
18. Pushko P et al. Virology 239(2):389401 (1997).
19. Tang et al., Nature 356: 152-154 (1992).
20. Zang D-J et al. J Infec Dis 176:1035-1040 (1997).

What I claim is:

1. An isolated and purified nucleic acid molecule comprising a nucleic acid sequence which encodes a polypeptide selected from any one of:
(a) SEQ ID No: 2;
(b) SEQ ID No: 4;
(c) SEQ ID No: 6;
(d) SEQ ID No: 8;
(e) an immunogenic fragment comprising at least 12 consecutive amino acids from a polypeptide of (a) to (d); and
(f) a polypeptide of (a), (b), (c) or (d) which has been modified by conservative amino acid substitution without loss of immunogenicity, wherein said modified polypeptide is at least 75% identical in amino acid sequence to the corresponding polypeptide of (a), (b), (c) or (d).

2. A isolated and purified nucleic acid molecule comprising a nucleic acid sequence selected from any one of:
(a) SEQ ID No: 1;
(b) SEQ ID No: 3;
(c) SEQ ID No: 5;
(d) SEQ ID No: 7;
(e) a sequence comprising at least 38 consecutive nucleotides from any one of the nucleic acid sequences of (a) to (d); and
(f) a sequence which encodes a polypeptide which has been modified by conservative amino acid substitution without loss of immunogenicity and which is at least 75% identical in amino acid sequence to the polypeptides encoded by SEQ ID No: 1, 3, 5, or 7.

3. A isolated and purified nucleic acid molecule comprising a nucleic acid sequence which is complementary to any one of the nucleic acid molecule of claim 1.

4. A nucleic acid molecule comprising a nucleic acid sequence which encodes a fusion protein, said fusion protein comprising a polypeptide encoded by a nucleic acid molecule according to claim 1 and an additional polypeptide.

5. The nucleic acid molecule of claim 4 wherein the additional polypeptide is a heterologous signal peptide.

6. The nucleic acid molecule of claim 4 wherein the additional polypeptide has adjuvant activity.

7. A nucleic acid molecule according to any one of claims 1 to 6, operatively linked to one or more expression control sequences.

8. A vaccine comprising a vector comprising a nucleic acid molecule which encodes a polypeptide selected from any one of:
(a) SEQ ID No: 2;
(b) SEQ ID No. 4;
(c) SEQ ID No: 6;
(d) SEQ ID No: 8;
(e) an immunogenic fragment comprising at least 100 consecutive amino acids from the polypeptide of any one of (a) to (d); and
(f) a polypeptide of any one of (a) to (e) which has been modified by conservative amino acid substitution; wherein said modified polypeptide is at least 90% identical in amino acid sequence to the corresponding polypeptide of any one of (a) to (e); wherein the nucleic acid molecule is either operatively linked to one or more control sequences for expression of the polypeptide in a mammalian or a bacterial cell; wherein the vaccine provides an immune response protective against disease caused by Chlamydia.

9. The vaccine of claim 8 wherein the vaccine optionally comprises an additional nucleic acid encoding an additional polypeptide which enhances the immune response to the polypeptide selected from any one of (a) to (f).

10. A pharmaceutical composition comprising a pharmaceutically acceptable carrier or diluent suitable for use in a vaccine and a nucleic acid molecule which encodes a polypeptide selected from any one of:
(a) SEQ ID No: 2;
(b) SEQ ID No. 4;
(c) SEQ ID No: 6;
(d) SEQ ID No: 8;
(e) an immunogenic fragment comprising at least 100 consecutive amino acids from the polypeptide of (a) to (d); and
(f) a polypeptide of any one of (a) to (e) which has been modified by conservative amino acid substitution without loss of immunogenicity; wherein said modified polypeptide is at least 90% identical in amino acid sequence to the corresponding polypeptide of any one of (a) to (e); wherein the nucleic acid molecule is operatively linked to one or more control sequences for expression of the polypeptide in a mammalian cell.

11. The pharmaceutical composition of claim 10 comprising a pharmaceutically acceptable carrier or diluent suitable for use in a vaccine and a nucleic acid molecule which encodes a polypeptide selected from any one of:
(a). SEQ ID No: 2;
(b) SEQ ID No. 4;
(c) SEQ ID No: 6;
(d) SEQ ID No: 8; and
(e) an immunogenic fragment comprising at least 100 consecutive amino acids from the polypeptide of (a) to (d).

12. The pharmaceutical composition of claim 10 comprising a pharmaceutically acceptable carrier or diluent suitable for use in a vaccine and a nucleic acid molecule which encodes a polypeptide selected from any one of:
(a) SEQ ID No: 2;
(b) SEQ ID No. 4;
(c) SEQ ID No: 6;
(d) SEQ ID No: 8; and
(e) a polypeptide of any one of (a) to (d) which has been modified by conservative amino acid substitution without loss of immunogenicity, wherein said modified polypeptide is at least 90% identical in amino acid sequence to the corresponding polypeptide of any one of (a) or (d).

13. The vaccine of claim 8 comprising a vaccine vector wherein the vaccine vector comprises a nucleic acid molecule which encodes a polypeptide selected from any one of:
(a) SEQ ID No: 2;
(b) SEQ ID No. 4;
(c) SEQ ID No: 6; and
(d) SEQ ID No: 8.

14. The vaccine of claim 8 comprising a vaccine vector wherein the vaccine vector comprises a nucleic acid molecule which encodes a polypeptide selected from any one of:
(a) SEQ ID No: 2;
(b) SEQ ID No. 4;
(c) SEQ ID No: 6;
(d) SEQ ID No: 8; and
(e) an immunogenic fragment comprising at least 100 consecutive amino acids from the polypeptide of (a) to (d).

15. The vaccine of claim 8 comprising a vaccine vector wherein the vaccine vector comprises a nucleic acid molecule which encodes a polypeptide selected from any one of:
(a) SEQ ID No: 2;
(b) SEQ ID No. 4;
(c) SEQ ID No: 6;
(d) SEQ ID No: 8; and
(e) a polypeptide of any one of (a) to (d) which has been modified by conservative amino acid substitution without loss of immunogenicity, wherein said modified polypeptide is at least 90% identical in amino acid sequence to the corresponding polypeptide of any one of (a) to (d).

16. A method for preventing or treating Chlamydia infection comprising the step of administering an effective amount of a nucleic acid molecule which encodes a polypeptide selected from any one of:
(a) SEQ ID No: 2;
(b) SEQ ID No: 4;
(c) SEQ ID No: 6;
(d) SEQ ID No. 8;
(e) an immunogenic fragment comprising at least 100 consecutive amino acids from the polypeptide of (a) to (d); and
(f) a polypeptide of any one of a) to (e) which has been modified by conservative amino acid substitution without loss of immunogenicity, wherein said modified polypeptide is at least 90% identical in amino acid sequence to the corresponding polypeptide of any one of (a) to (e); wherein the nucleic acid molecule is operatively linked to one or more control sequences for expression of the polypeptide.

17. The method of claim 17 for preventing or treating Chlamydia infection, comprising the step of administering an effective amount of a nucleic acid molecule which encodes a polypeptide selected from any one of:
(a) SEQ ID No: 2;
(b) SEQ ID No: 4;
(c) SEQ ID No: 6; and
(d) SEQ ID No. 8.

18. The method of claim 17 for preventing or treating Chlamydia infection, comprising the step of administering an effective amount of a nucleic acid molecule which encodes a polypeptide selected from any one of:
(a) SEQ ID No: 2;
(b) SEQ ID No: 4;
(c) SEQ ID No: 6;
(d) SEQ ID No. 8; and
(e) an immunogenic fragment comprising at least 100 consecutive amino acids from the polypeptide of (a) to (d).

19. The method of claim 17 for preventing or treating Chlamydia infection, comprising the step of administering an effective amount of a nucleic acid molecule which encodes a polypeptide selected from any one of:
(a) SEQ ID No: 2,
(b) SEQ ID No: 4;
(c) SEQ ID No: 6;
(d) SEQ ID No. 8; and
(e) a polypeptide of any one of (a) to (d) which has been modified by conservative amino acid substitution, wherein said modified polypeptide is at least 90% identical in amino acid sequence to the corresponding polypeptide of any one of (a) or (d).

20. A unicellular host transformed with the nucleic acid molecule of claim 7.

21. A nucleic acid probe of 5 to 100 nucleotides which hybridizes under stringent conditions to the nucleic acid molecule of SEQ ID No: 1, 3, 5 or 7, or to a homolog or complementary oranti-sense sequence of said nucleic acid molecule.

22. A primer of 10 to 40 nucleotides which hybridizes under stringent conditions to the nucleic acid molecules of SEQ ID No: 1 or 3, or to a homolog or complementary or anti-sensesequence of said nucleic acid molecule.

23. A polypeptide encoded by a nucleic acid sequence according to any one of claims 1, 2 and 4 to 7.

24. A method for producing a polypeptide of claim 7 comprising the step of culturing a unicellular host according to claim 21.

25. An antibody against the polypeptide of any one of claims 24.

26. A vaccine comprising at least one first polypeptide according to any one of claims 1, 4, to 7 and a pharmaceutically acceptable carrier, optionally comprising a second polypeptide which enhances the immune response to the first polypeptide.

27. The vaccine of claim 27 wherein the second polypeptide comprises an additional Chlamydia polypeptide.

28. A pharmaceutical composition comprising a polypeptide according to any one of claims 1, 4 to 7 and a pharmaceutically acceptable carrier.

29. A pharmaceutical composition comprising a vaccine according to claim 27 or 28 and a pharmaceutically acceptable carrier.

30. An isolated polynucleotide from a strain of Chlamydia selected from the group consisting of:
(a) a polynucleotide comprising the nucleotide sequence of SEQ ID NO:1;
(b) a polynucleotide comprising the nucleotide sequence of SEQ ID NO:3;
(c) a polynucleotide comprising the nucleotide sequence of SEQ ID NO:5;
(d) a polynucleotide comprising the nucleotide sequence of SEQ ID NO:7;
(e) a polynucleotide that is at least 95% homologous to the nucleotide sequence of SEQ ID NO:1, 3, 5, or 7; and
(f) a polynucleotide which hybridizes under stringent hybridizing conditions of 6×SSC containing 50% formamide at 42° C. with a polynucleotide comprising the nucleotide sequence of SEQ ID NO: 1, 3, 5, or 7;
wherein administration of said isolated polynucleotide, in an immunogenically-effective amount to a mammal, induces an immune response in said mammal against infection by said strain of Chlamydia.

31. An isolated and purified polypeptide molecule comprising a polypeptide selected from any one of:
(a) SEQ ID No: 2;
(b) SEQ ID No: 4;
(c) SEQ ID No: 6;
(d) SEQ ID No: 8;
(e) an immunogenic fragment comprising at least 12 consecutive amino acids from a polypeptide of (a) to (d); and
(f) a polypeptide of (a), (b), (c) or (d) which has been modified by conservative amino acid substitution without loss of immunogenicity;
wherein said modified polypeptide is at least 75% identical in amino acid sequence to the corresponding polypeptide of (a), (b), (c) or (d).

32. A polypeptide molecule of claim 31 further comprising a heterologous signal peptide.

33. A vaccine comprising a polypeptide selected from any one of:
(a) SEQ ID No: 2;
(b) SEQ ID No. 4;
(c) SEQ ID No: 6;
(d) SEQ ID No: 8;
(e) an immunogenic fragment comprising at least 100 consecutive amino acids from the polypeptide of any one of (a) to (d); and
(f) a polypeptide of any one of (a) to (e) which has been modified by conservative amino acid substitution, wherein said modified polypeptide is at least 90% identical in amino acid sequence to the corresponding polypeptide of any one of (a) to (e); wherein the nucleic acid molecule is either operatively linked to one or more control sequences for expression of the polypeptide in a mammalian or a bacterial cell, wherein the vaccine provides an immune response protective against disease caused by Chlamydia

34. A pharmaceutical composition comprising a pharmaceutically acceptable carrier or diluent suitable for use in a vaccine and a polypeptide selected from any one of:
(a) SEQ ID No: 2;
(b) SEQ ID No. 4;
(c) SEQ ID No: 6;
(d) SEQ ID No: 8;
(e) an immunogenic fragment comprising at least 100 consecutive amino acids from the polypeptide of (a) to (d); and
(f) a polypeptide of any one of (a) to (e) which has been modified by conservative amino acid substitution without loss of immunogenicity;
wherein said modified polypeptide is at least 90% identical in amino acid sequence to the corresponding polypeptide of any one of (a) to (e).

35. The vaccine of claim 33 further comprising an adjuvant.

36. The vaccine of claim 35 wherein said adjuvant is an ISCOM adjuvant.

37. The pharmaceutical composition of claim 34 comprising a pharmaceutically acceptable carrier or diluent suitable for use in a vaccine and a nucleic acid molecule which encodes a polypeptide selected from any one of:
(a) SEQ ID No: 2;
(b) SEQ ID No. 4;
(c) SEQ ID No: 6;
(d) SEQ ID No: 8; and
(e) an immunogenic fragment comprising at least 100 consecutive amino acids from the polypeptide of (a) to (d).

38. A method for preventing or treating Chlamydia infection comprising the step of administering an effective amount of a polypeptide selected from any one of:
(a) SEQ ID No: 2;
(b) SEQ ID No: 4;
(c) SEQ ID No: 6;
(d) SEQ ID No. 8;
(e) an immunogenic fragment comprising at least 100 consecutive amino acids from the polypeptide of (a) to (d); and
(f) a polypeptide of any one of (a) to (e) which has been modified by conservative amino acid substitution without loss of immunogenicity; wherein said modified polypeptide is at least 90% identical in amino acid sequence to the corresponding polypeptide of any one of (a) to (e).
